(19) United States
(12) Patent Application Publication
(10) Pub. No.: US 2013/0130304 A1
(43) Pub. Date: May 23, 2013

(54) METHODS FOR SCREENING MICROBIAL GROWTH INHIBITION ACTIVITY ON MATERIALS

(71) Applicant: Saint Louis University, a non-profit organizaton, St. Louis, MO (US)

(72) Inventors: Anupma Dixit, St. Louis, MO (US); Brunda Tumala, Chesterfield, MO (US)

(73) Assignee: Saint Louis University, a non-profit organizaton, St. Louis, MO (US)

(21) Appl. No.: 13/683,948

(22) Filed: Nov. 21, 2012

Related U.S. Application Data
(60) Provisional application No. 61/563,390, filed on Nov. 23, 2011.
(60) Provisional application No. 61/563,375, filed on Nov. 23, 2011.

(51) Int. Cl. C12Q 1/18

(52) U.S. Cl. CPC C12Q 1/18
USPC 435/32

(57) ABSTRACT

Disclosed are methods of determining the effectiveness of an antimicrobial agent in reducing or inhibiting microbial growth on a substrate. The disclosed methods may be used to determine microbial growth at time points subsequent to antimicrobial treatment of the material surface and exposure to microorganisms. The disclosed invention also measures visible microbial growth using a semi-quantitatively method that is more accurate and less subjective than estimates of growth used previously. The disclosed method that provides an accurate assessment of antimicrobial efficacy with reduced variability.

### CROSS REFERENCE TO RELATED APPLICATIONS

This application claims priority to provisional application 61/563,375, filed Nov. 23, 2011, and provisional application 61/563,390 filed Nov. 23, 2011, both of which are hereby incorporated by reference in their entirety.

### GOVERNMENT SUPPORT CLAUSE

This work was supported by HUD grants MOLHH0167-08 and MOLHH0195-09. The U.S. Government has certain rights in this invention.

### FIELD OF THE INVENTION

The invention relates to methods and compositions for testing the antimicrobial activities of known and experimental antimicrobial formulations for the purposes of determining the effectiveness of an antimicrobial agent in reducing or inhibiting microbial growth on a substrate.

### BACKGROUND

Damp buildings are associated with a spectrum of adverse health outcomes in sensitive individuals (Institute of Medicine. (2004) The National Academies Press, Washington, D.C., and World Health Organization (2009) ISBN 798 92 890 4168 3, Publications; WHO Regional Office for Europe, Copenhagen, Denmark). Fungal (mold) growth on indoor surfaces following moisture incursion represents a potential health hazard for the building occupants. A US cost estimate attributable to indoor dampness and mold exposure for asthma alone was $3.5 billion (Mudarri, and Fisk (2007) Indoor Air. 17:226-235) Gypsum dry wallboard is commonly used as an indoor construction material, providing perhaps the largest surface area of any one kind inside a typical residential building. After moisture exposure, gypsum dry wallboard and other building and furnishing materials are prone to fungal colonization. Most mold remediation guidelines agree that moldy or permeable and semipermeable materials wet for more than 24 hours must be discarded (Cole and Foarde (1999) Biocides and Antimicrobial Agents. In: J. Macher (ed.). Bioaerosols: Assessment and Control. American Conference of Governmental and Industrial Hygienists, Cincinnati, Ohio). Nonetheless, there is a growing recognition that sometimes it is not done or there are situations where discarding is not always feasible or desirable (Price and Ahearn (1999) Curr. Microbiol. 39: 21-26.; Krause et al., (2006) J. Occup. Environ. Hyg. 3:435-441; Menetrez, et al., (2008) J. Occup. Environ. Hyg. 5:63-66). To date, only a small number of studies have examined the effectiveness of common surface treatment methods such as cleaning of moldy dry wallboard by dry brushing, wiping, and/or treatment with chemicals, including antimicrobial coatings (Id.). The current method recommended for testing fungistatic resistance of antimicrobial paints and coating films is ASTM D-5590, Standard Test Method for Determining the Resistance of Paint Films and Related Coatings to Fungal Defacement by Accelerated Four-Week Agar Plate Assay (ASTM D-5590 (2005) American Society for Testing and Materials. West Conshohocken, Pa.). This method utilizes filter paper as a substrate which is loaded with the test agent. The substrate is inoculated with a cotton swab, pipette or atomizer and placed in an agar dish and maintained at 28° C., with relative humidity of 85-90 percent. Assessment is in the form of a qualitative (0-4) determination of visible fungus after 4 weeks.

The current method suffers from several short comings. It does not take into account the physical differences between building materials or environmental requirements of a broad range of microbes. Building materials differ with respect to composition and porosity which directly influences the rate of absorption, diffusion, and/or leaching of antimicrobials both during and after treatment. Building materials also provide a range of growth environments for microbes. It is not possible to simulate all of these factors with methods based on filter paper. In addition, not all microbes will grow in relative humidity of 85-90 percent as used in current methods. The disclosed method can be used to test formulations against bacterial or fungi differing in morphology, nutrition, as well as substrate temperature and moisture requirements. The use of building materials as substrates allows for the determination of antimicrobial activities of experimental formulations on a wide range of microbes with varied nutritional and environmental requirements, and is not limited to those that grow only on filter paper under high humidity.

In addition, existing methods such as ASTM D-5590 determine growth by visual observation, and require at least several days for mold to grow on a substrate before it can be assessed. The Inventors discovered that even if there is no visible fungal growth on a substrate, viable fungal elements may remain which can be detected only by extraction of the substrate and subsequent culture to determine colony counts. The disclosed methods may be used to determine microbial growth at time points subsequent to antimicrobial treatment of the material surface treatment and exposure to microorganisms. In addition, the disclosed invention also measures visible growth semi-quantitatively instead of a subjective visual estimate approach. By determining the percent of the substrate area covered with microbes using for example, a quantitative grid, data obtained can be then converted to scores based on a 0-5 rating system. The result is more accurate and less subjective than estimates of growth used previously.

The inventors also discovered the importance of evenly dispersing and controlling microbial inoculations. This reduces variation between replicates and treatments. Previous methods sprayed an unspecified volume (thin-coat) of microbial suspension onto substrates and surrounding agar which did not result in even-distribution of microbes or a known number. Consequently, microbe concentrations differed across the substrate and the surrounding culture medium.

The inventors have discovered a method by which to test a broad range of antimicrobials on building materials, finishing, and furnishing materials under a broad range of environmental conditions. In addition, they have discovered a method that has reduced variability and arrives at a more accurate assessment of antimicrobial efficacy.

### SUMMARY OF THE INVENTION

A method of determining the effectiveness of an antimicrobial agent in reducing or inhibiting microbial growth on a substrate, comprising:
a) treating a substrate with the antimicrobial agent,
b) dispersing a volume of the microbial suspension of known concentration on the top surface of the substrate,
c) placing the substrate in a sterile culture container,
d) incubating the substrate for one or more periods of time under conditions that will allow growth of the microbe,
e) determining viable colony forming units by extracting viable microbes from the substrate and plating on nutritive media and incubating under conditions which allow growth of the microbes and,
f) comparing the viable colony forming units obtained from substrates treated with the antimicrobial agent to either substrates not treated with an antimicrobial agent or to substrates treated with a different antimicrobial agent and subjected to the same conditions

A method of determining the effectiveness of an antimicrobial agent in reducing or inhibiting microbial growth on a substrate, comprising:
a) placing the substrate in the center of an agar plate, the agar plate comprising nutritive culture medium and inoculated with a microbial suspension of known concentration,
b) evenly dispersing a volume of the microbial suspension of known concentration on the top surface of the substrate,
c) incubating the substrate for period of at a temperature and relative humidity that will allow growth of the microbe,
g) determining a zone of inhibition around the substrate by measuring the area of microbial growth inhibition around the substrate and,
d) comparing the zone of inhibition obtained from substrates treated with the antimicrobial agent to either a zone of inhibition obtained from substrates not treated with a antimicrobial agent or substrates treated with a different antimicrobial agent and subjected to similar conditions.

A method of determining the effectiveness of an antimicrobial agent in reducing or inhibiting microbial growth on a substrate, comprising:
a) treating a substrate with the antimicrobial agent,
b) incubating the substrate for one or more periods of time, in the field at a test site that will allow growth of one or more microbes,
c) determining viable colony forming units by extracting viable microbes from the substrate and plating the extract on nutritive media under conditions which allow growth of the microbes and,
d) comparing the viable colony forming units obtained from the substrates treated with the antimicrobial agents to viable colony forming units obtained from substrates not treated with an antimicrobial agent or to viable colony forming units obtained from substrates treated with a different antimicrobial agent and subject to similar conditions.

### DESCRIPTION OF THE FIGURES

FIG. 1 illustrates a testing protocol utilizes building materials as substrates and residual viability testing.

FIG. 2 illustrates a testing protocol that utilizes building material substrates, residual viability testing, a Zone of inhibition assay and assesses substrate surface growth.

FIG. 3 illustrates an example of a treated substrate in a Zone of inhibition assay.

FIG. 4 illustrates the results of residual viability testing after a 24 hour incubation period with treated substrates. Error bars indicate average (±SD) percent log reduction in fungal viability.

FIG. 4.1 illustrates one alternative method of expressing the data shown in FIG. 4, where the average log reduction or geometric mean reduction±SD (geometric standard deviation) in fungal viability after each antimicrobial agent is compared to a water control.

FIG. 5 illustrates the results of residual viability testing after a 5 week incubation period with treated substrates. Error bars indicate average (±SD) percent log reduction in fungal viability.

FIG. 5.1 illustrates one alternative method of expressing the data shown in FIG. 5, where the average log reduction or geometric mean reduction±SD (geometric standard deviation) in fungal viability after each antimicrobial agent is compared to a water control.

FIG. 6 illustrates average (±SD) Zones of Inhibition (ZOI) for A. versicolor over an incubation period of 5 weeks using gypsum wallboard as a substrate.

FIG. 7 illustrates average (±SD) percent visible growth of A. versicolor on gypsum wallboard substrate over an incubation period of 5 weeks.

FIG. 8 illustrates average (±SD) Zones of Inhibition (ZOI) for S. chartarum over an incubation period of 5 weeks using gypsum wallboard as a substrate.

FIG. 9 illustrates average (±SD) percent visible growth of S. chartarum on gypsum wallboard substrate over an incubation period of 5 weeks.

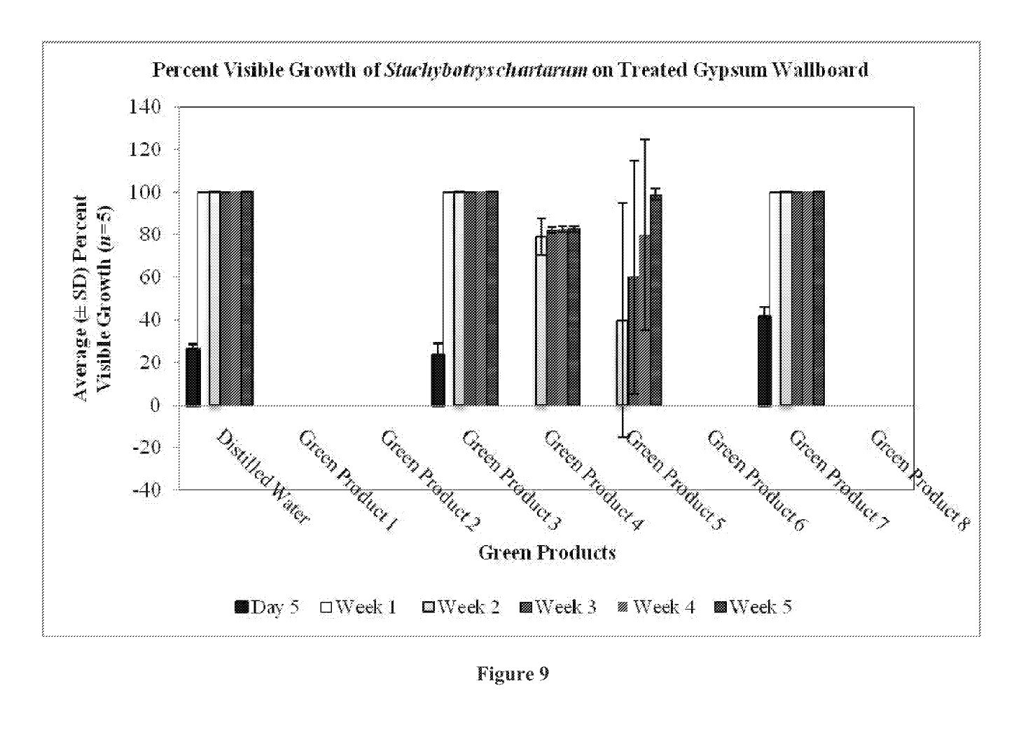

FIG. 10 illustrates average (±SD) percent log reduction in viability of A. versicolor and S. chartarum after 24 hours incubation with treated substrates.

FIG. 10.1 illustrates one alternative method of expressing the data shown in FIG. 10, where the average log reduction or geometric mean reduction±SD (geometric standard deviation) in fungal viability after each antimicrobial agent is compared to a water control.

FIG. 11 illustrates Average (±SD) log reduction in viability of A. versicolor and S. chartarum after a 5 week incubation period with treated substrates.

FIG. 11.1 illustrates one alternative method of expressing the data shown in FIG. 1, where the average log reduction or geometric mean reduction±SD (geometric standard deviation) in fungal viability after each antimicrobial agent is compared to a water control.

FIG. 12 illustrates average (±SD) Zones of inhibition for A. alternata over a period of 8 weeks using gypsum wallboard as a substrate.

FIG. 13 illustrates average (±SD) percent visible substrate surface growth of A. alternata on gypsum wallboard substrate over an incubation period of 8 weeks.

FIG. 14 illustrates average (±SD) Zones of inhibition for P. brevicompactum over an incubation period of 8 weeks using plywood as a substrate.

FIG. 15 illustrates average (±SD) percent visible substrate surface growth of P. brevicompactum on plywood substrate over an incubation period of 8 weeks.

FIG. 16 illustrates average (±SD) percent log reduction in viability of A. alternata and P. brevicompactum after 24 hours incubation with a treated substrate.

FIG. 16.1 illustrates one alternative method of expressing the data shown in FIG. 16, where the average log reduction or geometric mean reduction±SD (geometric standard deviation) in fungal viability after each antimicrobial agent is compared to a water control.

FIG. 17 illustrates average (±SD) percent log reduction in viability of Alternaria alternata and P. brevicompactum after 8 weeks incubation with treated substrate.

FIG. 17.1 illustrates one alternative method of expressing the data shown in FIG. 17, where the average log reduction or geometric mean reduction±SD (geometric standard deviation) in fungal viability after each antimicrobial agent is compared to a water control.

### DETAILED DESCRIPTION

Disclosed herein are methods for determining the efficacy of antimicrobial agents used in the treatment or coating or building materials to prevent microbial growth. The methods utilize building materials that are representative of those used in the field. The term building material is meant to include any and all materials that may be used in building construction, including materials used in building furnishing, and building finishing. The disclosed methods may be used with any material for which it is desirable to treat that material with antimicrobials. The samples, coupons, or carriers of building materials used in the test methods and examples disclosed herein are referred to collectively as substrates. Any material used in building construction or building furnishing may be used as a substrate. Non-limiting examples of building materials include: nonporous materials, including hard surface materials; semiporous materials including drywall, engineered wood, wood, particle board, oriented strand board, and framing lumber; flooring materials, including carpet, carpet padding, wood, and laminate; as well as porous materials, including insulation, textile, and synthetic material furnishings.

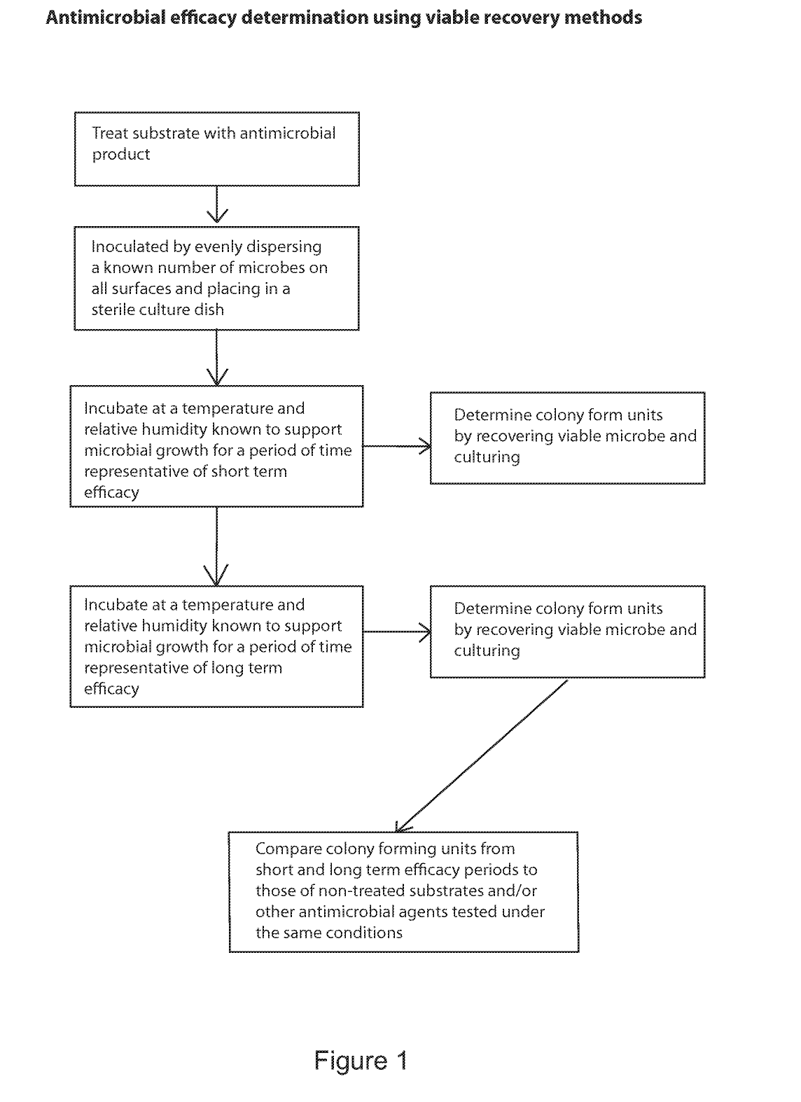

Substrates are cut into a convenient size, and treated with the particular antimicrobial agent to be tested. Alternatively, substrates may be cut to a convenient size to assist in handling, or for assessments based on surface area at any convenient time prior to analysis. Any method of applying the antimicrobial treatment may be utilized. A preferred method is that method which is recommended by the manufacturer for the particular antimicrobial agent and the substrate. By way of non-limiting example, a 2×2×0.5 inch dry wall, engineered wood, or carpeting may be sprayed, immersed, or painted with an antimicrobial according to the manufacturer's recommendations. Substrates may be sterilized, preferable before being treated. In one embodiment, as illustrated by one example in FIG. 1, sterile substrates are treated with an antimicrobial and then inoculated with test microbes by evenly dispersing a known suspension of test microbes on the top surface of the substrate. A suspension of test microbes may contain one or more different species of microbes. The substrate is then incubated in sterile conditions at a moderate temperature and relative humidity (RH), for example 25° C. and 65% RH, or a temperature and RH known to be supportive or optimum for the test microbes. The substrates may then be incubated for one or more periods of time representative of antimicrobial efficacy. By way of example, the substrates may be incubated for periods of time of about 24 hours and/or about 5 weeks, or periods of time representative of short term efficacy and/or long term efficacy of the antimicrobial agent. After each time period, viable microbes may be recovered and cultured in nutrient media to determine colony forming units. Colony forming units may be determined by any number of methods known in the art. By way of example, substrates may be individually placed inside sterile bags or containers and washed with a known volume of a sterile aqueous solution, for example phosphate buffered saline, and microbes extracted for several minutes or hours, preferably with agitation. After appropriate dilution, resultant extracts may be plated on nutritive media, for example agar plates and the number of colonies determined after an appropriate period of time, for example 1 day to 10 days, depending on the test microbe. By way of general example, bacteria may typically require about 24 hours whereas fungi may typically require 2 days or longer before colonies may be detected visually without the aid of a microscope. The number of colony forming units may be expressed as microbes per milliliter in the original extract solution and/or may be used to determine the number of colony forming units per unit area of the substrate. A greater reduction in colony forming units per milliliter or per unit area will indicate greater efficacy at that particular time point.

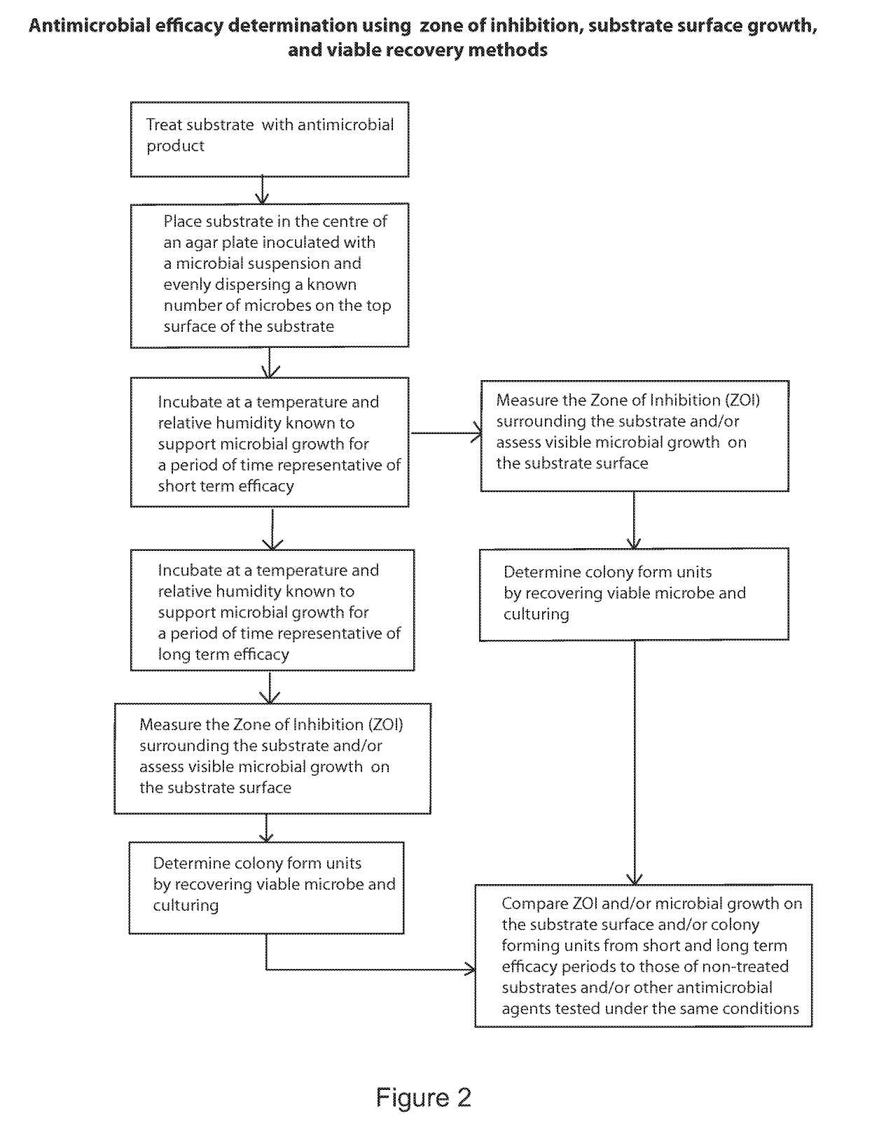

In another embodiment, as illustrated by one example in FIG. 2, sterile substrates are treated with an antimicrobial agent and then placed in an agar containing culture plate which has been inoculated with an evenly dispersed microbial suspension of known concentration, or a known number, of one or more test microbes. The microbial suspension is then evenly dispersed on a top surface of the substrate and the substrate is incubated at a temperature and relative humidity known to be supportive or optimum for the one or more test microbes. The substrates may then be incubated for one or more periods of time representative of antimicrobial efficacy. By way of non-limiting example, the substrates may be incubated for periods of time of about 24 hours and/or about 5 weeks, or any period of time representative of short term efficacy and/or long term efficacy of the antimicrobial agent. After each time period, a zone of inhibition (ZOI) surrounding the substrate may be determined, preferably by measuring the area of inhibition or absence of visible microbial growth on the agar surface surrounding the substrate. Optionally, or in addition to determining ZOI after each time-period, viable microbes may be recovered from the substrate and cultured in nutrient media to determine colony forming units.

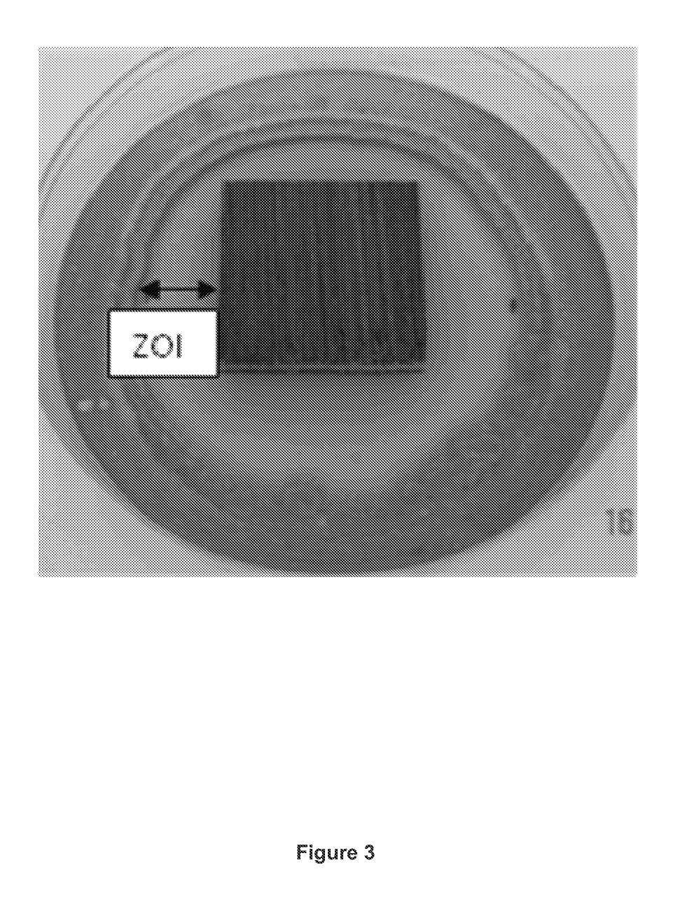

The Zone of Inhibition (ZOI) may be determined nondestructively by any number of means that determines the area of inhibition or absence of microbial growth surrounding the substrate. The area of inhibition or absence of microbial growth is determined visually, without the aid of microscopy, by comparing the surface of the agar surrounding the substrate to areas on the agar surface where a visible boundary of unaffected microbial growth is apparent (see FIG. 3) Non-limiting examples of determining the ZOI include measuring the distance from the edge of substrate to the visible boundary of unaffected microbial growth. A preferred, example of measuring the ZOI includes measuring the radius, or diameter of the area of inhibition or absence of microbial or growth surrounding the substrate. Measurement of the area of microbial inhibition or absence of microbial growth around the substrate may also be performed or aided using any variety of image analysis tools, including purpose designed image analysis software and digital photograph.

In addition to measuring the ZOI, microbial growth on the substrate surface may also be assessed nondestructively. By way of a preferred example, the area of visible microbial growth on the surface of the substrate may be measured by placing a grid over the culture dish and counting the number of squares occupied by microbial growth on the surface of the substrate. The area of growth on the substrate surface may be expressed as a percentage of the total area. Alternatively, once the area of microbial growth has been measured, the degree of microbial growth on the surface of the substrate may be scored using the criteria are set forth in Table 1.

TABLE 1
Rating Scheme for Visible Growth on Substrate Surfaces

| Visible Percent | |
|---|---|
| Growth | Rating |
| 75-100% | 5 |
| 50-74% | 4 |
| 30-49% | 3 |
| 15-29% | 2 |
| 5-14% | 1 |
| <5% Traces of | 0 |
| Growth | |

It is fully apparent that microbes grow on building materials and building furnishings in moist environments in the field. Therefore, another embodiment of the invention is to measure antimicrobial efficacy in the field using the natural environment to provide test microbes, as well as the culture conditions, including temperature and relative humidity. This embodiment would incorporate the determination of colony forming units as illustrated in FIG. 1, while utilizing the naturally occurring microbes and culture conditions that are available at a test site designated in the field. It is expected that growth or inhibition of the microbes used to determine efficacy in the field will be determined by comparing substrates incubated under similar or identical conditions at the test site, for example, treated substrates and non-treated substrates may be incubated adjacent to one another at the same test site or equivalent test sites in the field.

Regardless of the particular embodiment of the invention, inhibition of microbial growth represents antimicrobial activity or efficacy. The results may be expressed in any number of methods known in the art. Methods of comparing treated samples to non-treated samples are well known in the art and are commonly referred to as the use of controls. By way of example, results of a particular antimicrobial treatment, may be compared to substrates, which did not receive a antimicrobial treatment, (or were treated with a control solution, by way of example water), but have been subject to the testing under the same conditions, in particular, the same test microbial, number or concentrations of test microbes, as well as the same temperature and RH, and methods of comparing colony forming units, ZOI, or visible growth on the substrate surface after the same short term and/or long term efficacy time periods. Growth or inhibition may be expressed as percentage of the growth or inhibition of microbes on the non-treated or control substrate. In another example, the relative efficacy of different antimicrobials may be directly compared by testing different antimicrobials using the same conditions, in particular, the same test microbial, number or concentrations of test microbes, as well as the same temperature and RH, and methods of comparing colony forming units, ZOI, or visible growth on the substrate surface after the same short term and/or long term efficacy time periods. Results from any of the described analysis may be expressed through any mathematical transformation including but not limited to log. Any statistical comparisons known in the art may be used to compare quantitative data. Non-limiting examples include t-Test, and ANOVA or General Linear Modeling followed by Post Hoc analysis. The p-value for the acceptable level of statistical significance in mean difference between treatments should be predetermined.

### Test Microbes

Large and diverse groups of microbes are known to colonize building materials that have become moist or water-damaged. Any microbe that may grow or be cultured on building materials, either singly or as a mixture of microbes, may be used as a test microbe Non-limiting examples include bacterial and fungal microbes found at American Type Culture Collection (ATCC) (P.O. Box 1549, Manassas, Va. 20108). By way of example, fungal strains that are known to colonize a variety of moist building materials include: Alternaria alternata (ATCC® 58868™); Chaetomium globosum (ATCC® 34507™); Cladosporium sphaerospermum (ATCC® 11293M); and Penicillium brevicompactum (ATCC® 9056™). Examples of most preferred test microbes include fungi that frequently colonize moist building materials, including gypsum wallboard and wood products including Alternaria alternata, Aspergillus versicolor (ATCC®16856™) and other Aspergillus spp., Chaetomium globosum, Cladosporium spp., Penicillium spp., Stachybotrys chartarum (ATCC®18541™) and Trichoderma spp. Non-limiting examples of nonpathogenic and/or pathogenic gram negative and gram positive bacteria including Bacillus subtilus, B. cereus, Streptomyces spp., Mycobacterium spp., Pseudomonas spp., Escherichia coli, and Methicillin Resistant Staphylococcus aureus (MRSA).

### Inoculation of Substrates

Any number of methods may be employed to inoculate substrates with test microbes provided they result in an evenly dispersed known number of microbes. Typically this requires adjusting the volume and/or number or concentration of test microbes in the inoculum or suspension and applying a known volume of inoculum evenly to the substrate or agar surface with a dispersal device. Examples of methods used to evenly disperse a known number of test microbes include pipetting a known volume of a known number of test microbes on to a surface of the substrate, or agar, and evenly dispersing the volume with a sterile glass rod across the surface. Evenly dispersing a known number of test microbes reduces variation between the replicates and treatments. In addition, the top surface of each substrate is evenly dispersed in the same manner, further reducing variability between replicates and treatments. In order to be consistent between groups, typically the same or equivalent suspension will be utilized as an inoculum and the volume adjusted according to the size of the substrate or the desired number of test microbes to be applied.

In one embodiment, substrates are placed in the field, typically a construction site, and inoculation occurs naturally, through growth and/or movement of microbes at the site selected for testing.

### Culture Conditions and Incubation Times

The disclosed invention utilizes techniques generally known to artisans in the field. These techniques are described in detail in numerous laboratory protocols, one of which is entitled, A Photographic Atlas for the Microbiology Laboratory, by Leboffe and Pierce, (3^{rd} ed., 2005, Morton Publishing Co. Engelwood, Calif.), hereby incorporated by reference in its entirety. One of ordinary skill in the art may determine culture conditions for a given test microbe that are supportive or optimal for the purposes of practicing the invention without undue experimentation. The selection of test conditions may be based on information available regarding the original source of isolation, substrate preference, and biodeteriogenic activity from American Type Culture Collection (ATCC), and/or any other source, of information available including the scientific literature.

In at least one embodiment it is envisioned that testing may take place in the field or at sites where building materials or furnishing materials are exposed to moisture and microbes before, during, and after construction. These are typically construction sites, or any site where a building may be built or potentially may be built Non-limiting examples include existing structures in flood zone or low lying areas and buildings subjected/prone to moisture incursion through onetime, chronic or catastrophic water events. It is well known that conditions at these sites will support microbial, including fungal growth.

Similarly, one of ordinary skill in the art may also determine incubation times that are representative of short term and long term efficacy of antimicrobial agents by taking into account physicochemical properties of the antimicrobial and/or the particular building material being tested. Incubation times representing short or long term efficacy are also referred to herein as incubations times, incubations periods, or, as in the examples, referred to in reference to time periods where substrates are in contact with, or exposure to microbes. Physicochemical properties of the antimicrobial, including active half-life, solubility, and leaching times from particular materials may be based on information available from the manufacturer and/or the scientific literature.

It is not necessary that the test conditions or incubation times be optimal to practice the invention. It is only necessary that the test conditions and incubation times support growth and remain consistent between treated and non-treated substrates, or between treatment groups being subject to comparison.

### Substrates

Substrates include any and all materials used in building, constructing, finishing, and furnishing buildings. Non-limiting examples of building materials include: nonporous materials, including hard surface materials; semiporous materials including drywall, engineered wood, wood, particle board, oriented strand board, and framing lumber; flooring materials, including carpet, carpet padding, wood, and laminate; as well as porous materials, including insulation, textile, and synthetic material furnishings.

### Antimicrobials

Antimicrobials or antimicrobial agents include any natural occurring or manufactured compound that possess microbial killing or growth inhibiting properties, including any compound with fungistatic, fungicidal, bacteriostatic or bactericidal activity that may be applied to a building or furnishing material. Non-limiting preferred examples of antimicrobials are listed in Tables 2, 3, and 6.

TABLE 2
Examples of chemical products with antimicrobial activity

| | | Ready To Use |
|---|---|---|
| | | (RTU)/ |
| | | Recommended |
| Chemical product | Active ingredients* | dilution |
| DOT | Disodium Octaborate | 15% w/v |
| | Tetrahydrate | |
| Chlorine Product 1 | Sodium Hypochlorite (5.7%) | 1:10 |
| (CP1) | | |
| Chlorine Product 2 | Chlorine dioxide (0.72%), | 1:4 |
| (CP2) | Didecyl dimethyl ammonium | |
| | chloride (0.4%) | |
| Chlorine Product 3 | Stabilized Chlorine dioxide | RTU |
| (CP3) | (<1%), Alkyl Dimethyl Benzyl | |
| | Ammonium Chloride (<1%), | |
| | Alkyl Dimethyl ethylbenzyl | |
| | Ammonium Chloride (<1%) | |
| Hydrogen Peroxide | Hydrogen Peroxide (<10%) | RTU |
| Product 1 (HP1) | | |
| Hydrogen Peroxide | 3-Propyl dimethyl octadecyl | 1:16 |
| Product 2 (HP2) | ammonium chloride (<1%), | |
| | Hydrogen Peroxide (<5.5%), | |
| | Ethylene glycol monobutyl ether | |
| | (<6%), | |
| Phenol Product 1 | Phenyl Phenol (0.22%), | RTU |
| (PP1) | Dimethyl benzyl ammonium | |
| | chloride monohydrate(0.74%) | |
| Phenol Product 2 | Phenol (1.56%) and Sodium | RTU |
| (PP2) | phenate (0.06%) | |
| Quaternary | Octyl decyl dimethyl ammonium | RTU |
| Ammonium | chloride (0.025%), Dioctyl | |
| Compound 1 | dimethyl ammonium chloride | |
| (Quat 1) | (0.01%), Didecyl dimethyl | |
| | ammonium chloride (0.0155), | |
| | Alkyl dimethyl benzyl | |
| | ammonium chloride (0.034%) | |
| Quaternary | Dialkyl Dimethyl Ammonium | 1:64 |
| Ammonium | Chloride (3.3%), Alkyl Dimethyl | |
| Compound 2 | Benzyl Ammonium Chloride | |
| (Quat 2) | (2.2%) | |
| Quaternary | 1-Dimethyl Benzyl Ammonium | 1:64 |
| Ammonium | Chloride (<2.25%), 2-Dimethyl | |
| Compound 3 | Ethylbenzyl Ammonium | |
| (Quat 3) | Chloride (<2.25%) | |

*Based on information available on product label, Material Safety Data Sheet, or Technical Data Sheet

### EXAMPLES

### Materials and Methods

These methods and materials were used in each of the following examples unless otherwise specified.

### Test Microbes

Fungal strains that are known to colonize a variety of moist building materials were tested for their ability to colonize test-building substrates under experimental conditions. The selection of experimental fungal species and strains was based on the information available regarding the original source of isolation, substrate preference and biodeteriogenic activity from American Type Culture Collection (ATCC), and from information available in the scientific literature. All experimental fungi are native to North America and represent genera that are primary, secondary, or tertiary colonizers with varying substrate water activity requirements. The experimental fungi included: Alternaria alternata (ATCC® 58868™); Chaetomium globosum (ATCC® 34507™); Cladosporium sphaerospermum (ATCC® 11293™); Penicillium brevicompactum (ATCC® 9056™), Aspergillus versicolor (ATCC® 16856™), and Stachybotrys chartarum (ATCC® 18541™) although it is anticipated that the method may be used with any strain with no or miner modification. Of particular interest are fungi that frequently colonize moist building materials, including gypsum wallboard and wood products. Alternaria alternata, Cladosporium spp., and Penicillium spp. are among the most common allergenic fungi that occur in indoor and outdoor environments.

### Substrates

Building materials for use as experimental substrates included gypsum dry wallboard (SHEETROCK BRAND—Sound Deadening Board#0II8I1099II00065IIIII2-Gypsum Panel Label: WB2248-8/4-2000. Manufacturer: United States Gypsum Co., 125S. Franklin St., Chicago, Ill. 60606-4678) and engineered wood (Plywood Sheeting (3-Ply Rtd Sheathing, MFG Model #132411)

### Effect of Antimicrobial Coatings

Zone of Inhibition Assay (ZOI). Efficacies of antimicrobial coatings to inhibit growth of experimental fungi were evaluated using a Agar Plate-Zone of Inhibition Assay method (see FIG. 3). For the purpose of the current study, fungistatic and fungicidal effects after a 24 hour incubation period with antimicrobial coatings were considered short-term efficacy and those after 5-week incubation periods were regarded long-term efficacy.

Inoculated substrates were incubated at constant and relatively moderate temperature and relative humidity (25° C. and 65% RH) conditions. Semi-quantitative Assessment of visible growth of experimental fungi on substrates was done after 5-weeks. Additionally, residual viable fungi were determined at the end of 24 hour and 5 week incubation periods.

Fungistatic effectiveness of antimicrobial coatings was examined using wallboard as a substrate for Alternaria alternata (A. alternata), Chaetornium globosum (C. gobosum), and Cladosporium sphaerospermum (C. sphaerospermum). Plywood was used as substrate for Penicillium brevicompactum (P. brevicompactum) as this strain did not grow on wallboard at either 65% or at higher humidity.

Spore suspensions of P. brevicompactum (5.2×10⁶ spores/mL), C. sphaerospermum (3.4×10⁴ spores/mL), C. globosum (1.1×10⁵ spores/mL+1.4×10⁵ hyphae/mL+2×10³ fruiting bodies/mL), and A. alternata (2.4×10⁴ spores/mL) were used to inoculate petri dishes containing appropriate nutritive media. P. brevicompactum and C. globosum were spread-plated on Potato Dextrose Agar (PDA), C. sphaerospermum on Potato Dextrose Yeast (PDY) extract agar, A. alternata on V8 Juice Agar. Subsequently, sterile-coated and untreated (control) substrates were placed in the center of the inoculated culture plates and challenged by carefully spreading 500 microliters (μL) of the respective fungal suspension on the top surface (front papered side of the gypsum wallboard and finished surface of the plywood). Plates were incubated at constant 25° C. and 65% relative humidity in a dynamic environmental chamber (KB024—from Darwin Chambers, Inc.). Zone of inhibition, the width (mm) of clearing on one side of the substrate due to the absence of fungal growth was measured weekly during the 5 week follow-up period. Fungal growth on substrates was also visually evaluated on a weekly basis. A plastic 36 square-grid (30 mm×30 mm) was used to assess the substrate surface area covered with vegetative and sporulating fungal mycelium in a nondestructive manner. Zone of inhibition (ZOI) is shown in FIG. 3 concomitant with no visible growth of P. brevicompactum on plywood substrate 5 weeks post treatment.

FIG. 3 shows zone of inhibition at 5 weeks when plywood substrate is pretreated with Coating 2 and exposed to P. brevicompactum. There is no visible fungal growth on substrate surface or the zone of inhibition. Percent visible growth was determined as the fraction of total area (out of 36 squares) of the substrate covered with the fungal growth and subsequently rated on a 0-5 scale as described in Table 1

Zone of inhibition was rated by using the following nomenclature: “−” represents no Zone of Inhibition, “++++” represents ZOI≧25 mm, “+++” represents ZOI 15 mm-25 mm, “++” represents ZOI 5 mm-15 mm, “+” represents <5 mm ZOI. “*” represents stable ZOI that lasts till the end of treatment period, “**’ represents ZOI that diminishes slowly over treatment time period, “***” represents no ZOI observed at the end of treatment period.

### Example 1

Substrate Treatment:

Antimicrobials (See table 3) were applied to substrates as follows. Gypsum wallboard and plywood cut into 30 mm×30 mm sized substrates were individually wrapped and autoclaved at 121° C. for 30 minutes. The sterile substrates were then evenly and sequentially coated with a thin layer of antimicrobial test products on front, back and all four sides with a sterile glass spreader. Coated substrates were air dried for 48 hours inside a biosafety cabinet (Level II) to maintain sterility. Substrate without treatment (blank) served as the negative control. Five replicates per treatment were used. Fungal inoculums were prepared by gently scraping off the spore-rich material from fungal growth on nutritive media after flooding the culture dish with 10 milliliters (mL) of sterile distilled water.

### Antimicrobials

The tested antimicrobial coatings are listed in Table 3. Selection of coatings was based on active ingredients and not for product label verification.

TABLE 3
Antimicrobial Agents

| Test | |
|---|---|
| Products | Active Ingredients |
| Coating 1 | Titanium dioxide[<25%], 2-Tetrachloroisophthalonitrile |
| | [0.49%] |
| Coating 2 | Barium compound (15%) with Propynyl Butyl Carbamate |
| | (17%) |
| Coating 3 | Barium compound [5-10%], Titanium dioxide [1-5%], |
| | Zinc oxide [1-5%], Amorphous Silica [1-5%], and |
| | Propylene glycol [0.1-1%] |
| Coating 4 | 3-Iodo-2 Propynyl Butyl Carbamate (1%) |
| Coating 5 | Organosilane (a nanotechnology based product) |

Recovery of Fungi and Determination of Residual Viability.

After 5 weeks of incubation, for recovery of viable fungi, and determination of colony forming units, substrates were individually placed inside sterile stomacher bags (Fisher Scientific, Prod#01-002-54) and extracted in 20 mL of the Phosphate Buffered Saline (PBS, pH 7.2) using a Stomacher lab blender (Seward Lab system, Stomacher®80) operated for 120 seconds at normal speed. Resultant extracts were plated (100 μL) on nutritive media after appropriate dilution to achieve countable viable colony forming units (CFU's). Viable colonies were counted starting day 3 and followed up to 10 days. Additionally, in order to test the short-term effect of antimicrobial coatings, 500 μL of fungal suspension was dispersed on the top surface of the coated substrates, and viable fungi extracted after 24 hours. Resultant extract was subsequently plated on nutritive culture media to enumerate viable CFU's after a 24 hour incubation period.

The log reductions in fungal viability was calculated based on viable CFU's/mL on untreated or negative control substrate using the following formula:

Log Reduction=Log of (Average No. of Viable Colonies/mL on Negative Control/No. of Viable Colonies/mL on Coating/treated Substrate)

Absence of viable CFU's represented 100% reduction in the viability or log reduction equal to the log value of viable CFU's on control (represented by “*” in graphs). Log reduction of 1.0 in mean CFU's compared to control was used as the cutoff value for the efficacy determination (Zhu, et al., (2007) Eye & Contact Lens. 33: 278-283; Urban et al., (2011) J. App. Microbiol. 110:675-687). Viability reductions of ≧1.0 represented a positive effect whereas reductions of <1.0 signified no effect or difference from the control.

An alternative method of analysis involves determining whether or not the antimicrobial agent is able to achieve a 3.0 or more log reduction in viability (CFU's mL−1) at the recommended “in-use” concentrations on contaminated surfaces, with or without prior cleaning (see Disinfection, Sterilization, and Preservation, ed. S. Block, Lippincott, Williams & Wilkins, 2001, hereby incorporated by reference).

Data Analysis.

The outcomes from the prevention testing are shown as visible growth rating on “0-5” and the zone of inhibition size in “+ to ++++” scale. Due to the qualitative aspect of these data, statistical analysis was not applicable.

In testing where the outcome was viability, colony-forming unit data was normalized by log transformation. Log reductions were changed to percent (%) log reductions in order to equalize data for statistical analysis. The comparison of % mean log reduction values or log reduction values for multiple treatments was accomplished by the analysis of variance using one-way ANOVA or General Linear Modeling (GLM) procedure (SPSS-17), followed by Post Hoc analysis with Games-Howell test. The predetermined p-value for the acceptable level of statistical significance in mean difference between treatments was −0.05.

Zone of inhibition and Visible Growth on Substrate Surface

Irrespective of the fungal species, three types of coating responses were discerned with respect to the zone of inhibition on agar and visible growth on substrates (Tables 4 and 5):

TABLE 4
Zone of Inhibition (ZOI)

| | A. | C. | C. | P. |
|---|---|---|---|---|
| Treatment | alternata | globosum | sphaerospermum | brevicompactum |
| Blank | − | − | − | − |
| Coating 1 | ++*** | +*** | ++** | ++** |
| Coating 2 | +++* | ++* | +++* | +++* |
| Coating 3 | ++* | ++* | +++* | +++* |
| Coating 4 | − | − | − | − |
| Coating 5 | +++*** | ++*** | ++** | ++** |

In reference to table 4,
“−” represents no Zone of Inhibition,
“++++” represents ZOI ≧25 mm;
“+++” represents ZOI 15 mm-25 mm,
“++” represents ZOI 5 mm-15 mm,
“+” represents <5 mm ZOI.
“*” represents stable ZOI that lasts till the end of treatment period,
“**” represents ZOI that diminishes slowly over treatment time period,
“***” represents no ZOI observed at the end of treatment period.

TABLE 5
Visible Growth on the Substrate Surface

| | A. | C. | C. | P. |
|---|---|---|---|---|
| Treatment | alternata | globosum | sphaerospermum | brevicompactum |
| Blank | 5 | 5 | 5 | 5 |
| Coating 1 | 0 | 0 | 0 | 0 |
| Coating 2 | 0 | 0 | 0 | 0 |
| Coating 3 | 0 | 0 | 0 | 0 |
| Coating 4 | 5 | 5 | 5 | 5 |
| Coating 5 | 1 | 1 | 0 | 0 |

In reference to table 5, Group #1: Coatings with large (>15 mm) and stable zones of inhibition that showed complete inhibition of growth of fungi on substrates (Coating 3 and Coating 4).

Group #2: Coatings showing medium-sized (>10 mm) zones of inhibition that decreased over a period of 5 weeks. These coatings allowed none or minimal visible fungal growth on substrates starting day 3 post exposure (Coating 1 and Coating 5). Effect was less stable compared to group #1.

Group #3: Coating with no zone of inhibition, displaying 100% visible growth on substrates by day 5, similar to untreated control substrates (Coating 4)

The results obtained with fungi on various test substrates were in most part comparable with minor differences. Evidently, surface characteristics of substrates may influence absorption and permeation of the chemical product and consequently influence effectiveness of the antimicrobial coatings.

### Residual Viability

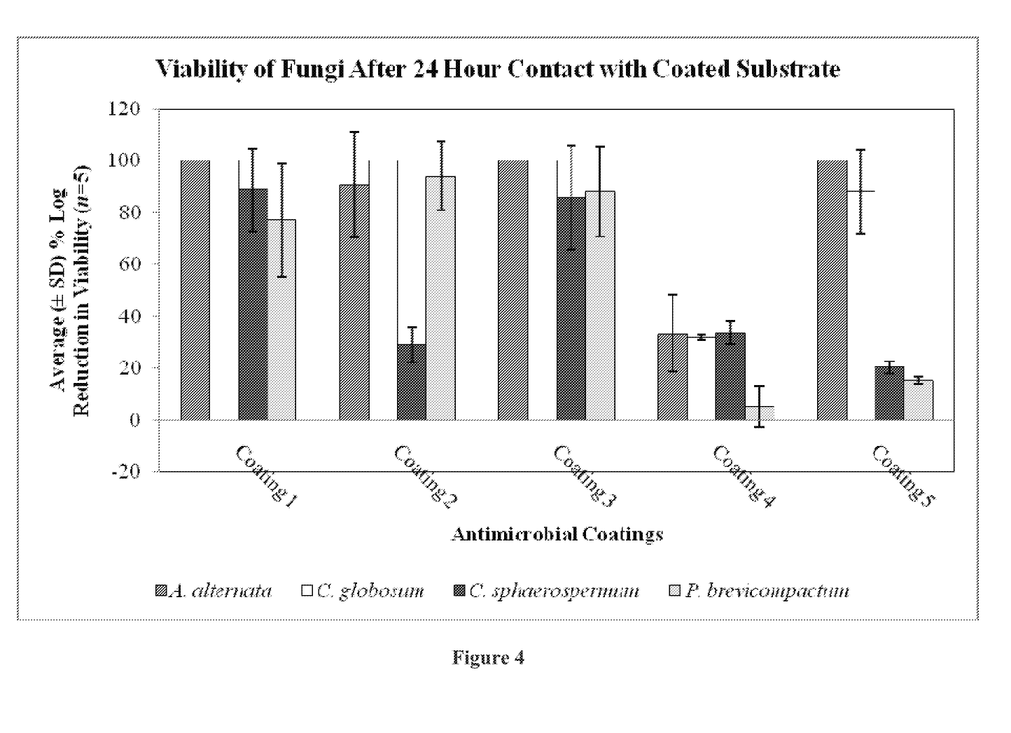

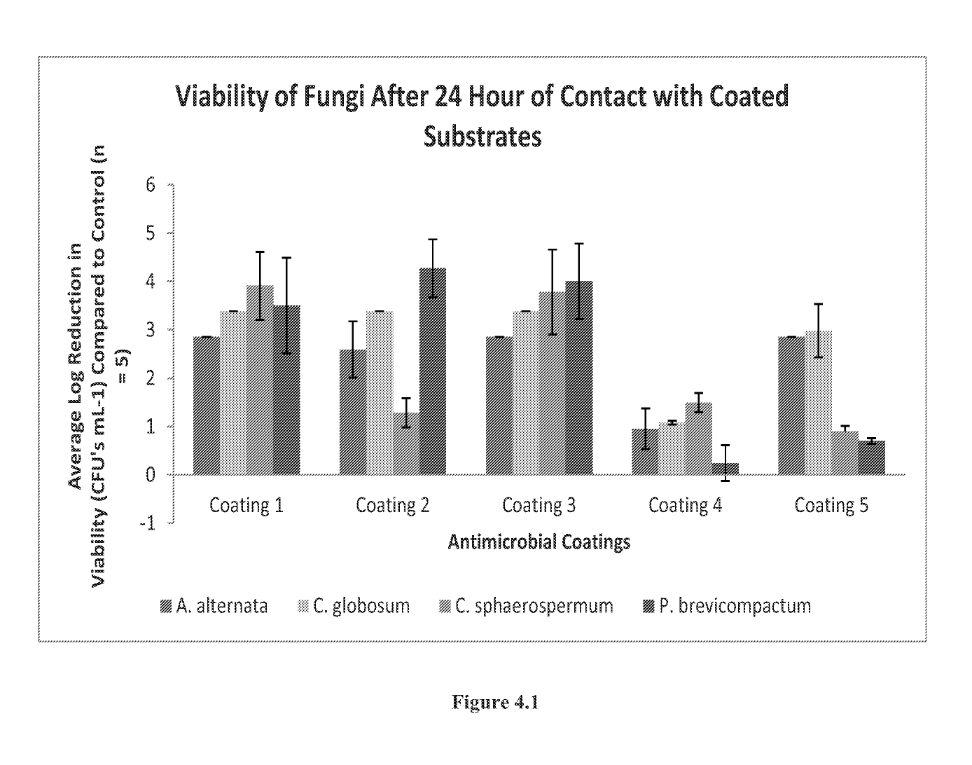

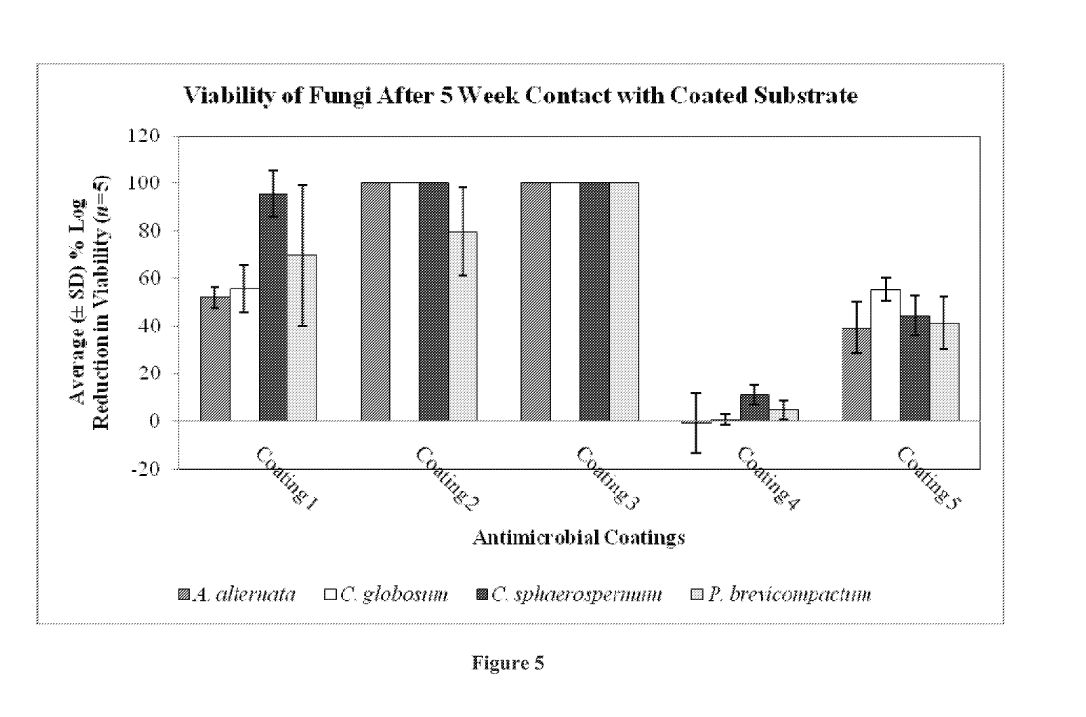

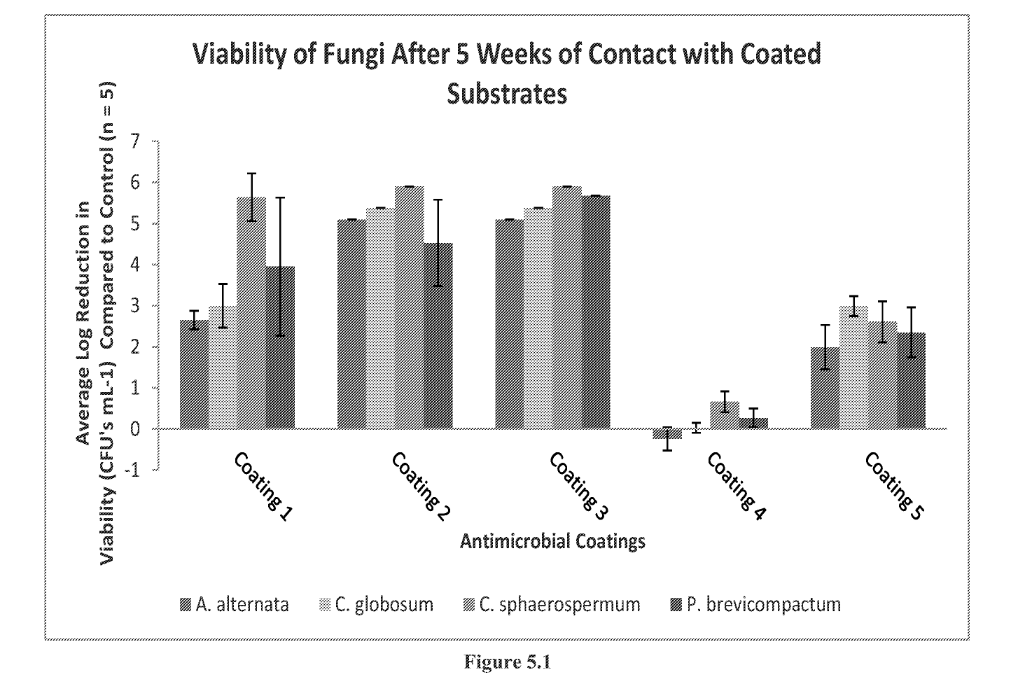

Viability reductions on treated substrates compared to the control untreated substrates indicate short and long-term effects of coatings after 24 hour and 5-week incubation periods, respectively (See FIGS. 4 and 5). A log reduction of 5.68 for P. brevicompactum, 5.9 for C. sphaeropermum, 5.38 for C. globosum, and 5.1 for A. alternata after 5 weeks' incubation represents 100% reductions. Log reductions of 4.54 for P. brevicompactum, 4.41 for C. sphaeropermum, 3.38 for C. globosum, and 2.89 for A. alternata after a 24 hour incubation period correspond to a 100% reduction.

An alternative method of expressing this data is shown in FIGS. 4.1 and 5.1 where average log reduction or geometric mean reduction±SD (geometric standard deviation) in fungal viability after antimicrobial agent compared to a water control.

The comparison of average percent log reductions in fungal viabilities after exposure of test fungi for 24 hours (FIG. 4) and 5 weeks (FIG. 5) to substrates pretreated with the five coatings showed that coatings 2 and 3 caused the highest decrease in the viability of most test fungi. No significant differences were seen in the reducing effect on individual genera (p-value=>0.05), except that the short-term efficacy of coating 2 on C. sphaerospermum, was marginal, differing significantly from decreases obtained with the remaining genera (p-value=<0.05). Coating 3 was the sole test product that invariably caused a 100% decrease in the long-term viability of all test fungi. Coating 2 totally mitigated viability of test fungi at long-term except for P. brevicompactum with high variability and lower reduction in viability after 5 weeks. For coating 1, significant differences were found in average % log reduction values after 5 weeks of exposure between the highest and nearly complete mitigation of C. sphaerospermum viability and decreases of close to half for A. alternata and C. globosum (p-value=<0.05). Differences in residual viability between other fungi and P. brevicompactum were not statistically significant due to a large variation in the data obtained for this fungus on plywood. Similarly, differences in the reducing effects on various test fungi and P. brevicompactum were not statistically significant in 24 hour assay (p-value=<0.05). Coating 4 did not have large effect on most test genera after 24 hours of exposure (Log reductions remained 1.5 compared to control). It was ineffective against all test genera in 5-week assay. Twenty four hour exposure subsequent to treatment with coating 5 resulted in 88% reduction and 100% reduction in the viabilities of C. globosum and A. alternata, respectively. The average % log reduction values for these two fungi were significantly different (p-value=<0.05) from those of the remaining two genera (C. sphaerospermum and P. brevicompactum) against which coating 5 was ineffective. No significant differences between fungi were discerned and relatively lower effect on A. alternata and C. globosum compared to decrease after a 24 hour incubation period was noted in the effect of Coating 5 in five weeks assay.

### Discussion of Results

The best performing formulations (Coating 2 and Coating 3), exhibiting large and stable zones of inhibition, show persistence and diffusion of active ingredients into the surrounding media from the point of contact. This feature is beneficial since the complete (100%) or nearly complete protection against colonization of diverse fungi after 5-weeks was observed in spite of variation in the short-term effect. These products provide excellent fungistatic and fungicidal activity against A. alternata, which is the most allergenic environmental fungal species in North America (Dixit et al., (2000) USA. GRANA. 39: 209-218). Incubation periods longer than 5 weeks may be necessary with the other test products showing lower efficacies against some of the test species. These products provide excellent fungistatic and fungicidal activity against A. alternata, which is the most allergenic environmental fungal species in North America (Id.). Coating 1 and Coating 5 with initially large but unstable zones of inhibition are variably effective but the effect on some genera fades away over time, suggesting rapid degradation of the active ingredients under certain conditions. Coating 5 is only about 50% effective after five weeks, irrespective of the target fungus. Thus, these two products are mostly suitable for situations where short-term prevention is desired.

The tested antimicrobial agents represent differing formulations, though some active ingredients are common, and most are EPA-registered. Three of the five coatings are mixtures of inorganic and organic chemicals of known fungicidal and/or insecticidal properties. Most notably, organic chemicals include 2-tetrachlorophthaonitrile (Coating 1), a polychlorinated aromatic compound and broad spectrum agricultural fungicide, also used as a preservative in paints and coatings, which can be degraded by some soil bacteria and fungi (Mori et al., (1998) Soil Sci. Plant Nutr. 44:297-304). Among the inorganic ingredients, titanium dioxide (Coating 1 and Coating 3) is a photo-catalyst that can oxidize organic material. It is added to paints and coatings to provide whiteness, for its sterilizing, deodorizing, anti-fouling properties. It is also used for waste-water remediation (Foster et al., (2011) Appl. Microbiol. Biotechnol. 90:1847-186). Zinc oxide (as in Coating 3) prevents corrosion of metals. Boron compounds (as in Coating 2 and Coating 3) are considered resistant to moisture and ultraviolet radiation. Barium borate is considered a broad-spectrum fungicide, insecticide, and a wood preservative. Among the single active ingredient formulations, Coating 5 contains carbamate, an agricultural fungicide, and Coating 4 is an organosilane, an organic compound containing carbon silicon bonds.

The two best performing coatings with long lasting effects in current investigation are multi-component formulations containing boron compounds it was also demonstrated previously that borate-based multi-component formulation is superior in prevention of diverse fungi compared to its individual components (Clausen and Yang (2007) Int. Biodeterior. Biodegrad. 59:20-24). Earlier studies with boron formulations noted cytotoxic and/or sporocidal effects with sodium polyborate-treated cellulose insulation on A. alternata, Aspergillus flavus, Aspergillus niger, Stachybotrys chartarum, and Cladosporium sphaerospermum (Herrea, (2005) J. Occup. Environ. Hyg. 2:626-632). Boron based coatings delayed (BORA-CARE) or inhibited (Foster 40-20) fungal growth on the pretreated gypsum wallboard (Krause et al., (2006) J. Occup. Environ. Hyg. 3:435-441; Menetrez, et al., (2008) Testing, J. Occup. Environ. Hyg. 5:63-66) reported prevention of Stachybotrys chartarum regrowth on gypsum wallboard by dry sponge wiping, and subsequent treatment with antimicrobial paints. A prior study involving wipe cleaning of Stachybotrys contaminated gypsum wallboard, followed by antimicrobial paint treatment with a variety of products, displayed no visible growth for six months with three (of seven) formulations (Id.) that contained active ingredients common to the best performing products in this study.

### Conclusions

Some antimicrobial coating products provide excellent protection against fungal colonization on pretreated semi-permeable building substrates. These are especially suitable for use on materials repeatedly exposed to high levels of moisture (e.g., in bathroom and kitchen) and in situations where water incursion remains a possibility (e.g., basements and lower floors of buildings in flood-prone areas).

By employing building materials as substrates, the disclosed method more accurately simulates field conditions including the absorption, permeation, and leaching of active ingredients into the surrounding media. It is observed that the results between ZOI and visible growth are consistent, and that outcomes obtained with multiple fungi on substrates are comparable. ZOI values obtained with coated building substrates were less variable than those obtained when filter paper was used as a substrate (data not shown). In addition the disclosed method is better at determining which antimicrobial agent is most effective for a particular microorganism.

### Example 2

Methods applied are the same as those described for Example 1 except where noted.

Substrate Treatment:

Antimicrobials designated as Green chemicals (See Table 6) were applied to substrates as follows. Gypsum wallboard, cut into 30 mm×30 mm sized substrates, were individually wrapped and autoclaved at 121° C. for 30 minutes. The sterile substrates were then pretreated with antimicrobials by immersing the substrates in 20 milliliters of antimicrobial formulation in a petridish for 10 minutes on each of the both surfaces. Treated substrates were used after draining the excessive liquid. Substrate immersed in distilled water (control) served as the non-treated or negative control. Czapek's agar was used for Aspergillus versicolor and Rabbit Food Agar as growth medium for Stachybotrys chartarum.

Green Mold Remediation Formulations

Eight commercial products labeled green by the manufacturer or distributor were tested against Aspergillus versicolor and Stachybotrys chartarum. The selected fungi represent toxigenic fungi that colonize wet building substrates.

TABLE 6
Green Antimicrobial Agents.

| ID # | Compound |
|---|---|
| 1 | Green Product 1 (0.23% Thymol) |
| 2 | Green Product 2 (Pure Australian Tea tree oil) |
| 3 | Green Product 3 (0.95% Sodium carbonate (soda ash) with |
| | patented blend of Inorganic compounds) |
| 4 | Green Product 4 (Water and unnamed plant based product) |
| 5 | Green Product 5 (30% ethanol) |
| 6 | Green Product 7 (Clove bud oil and unnamed essential oils) |
| 7 | Green Product 6 (Natural blend of enzymes, fermented byproducts |
| | of sugarcane and vegetables & citric acid) |
| 8 | Green Product 8 (blend of clove flower bud, lemon rind, |
| | cinnamon bark, eucalyptus leaf, rosemary leaf) |

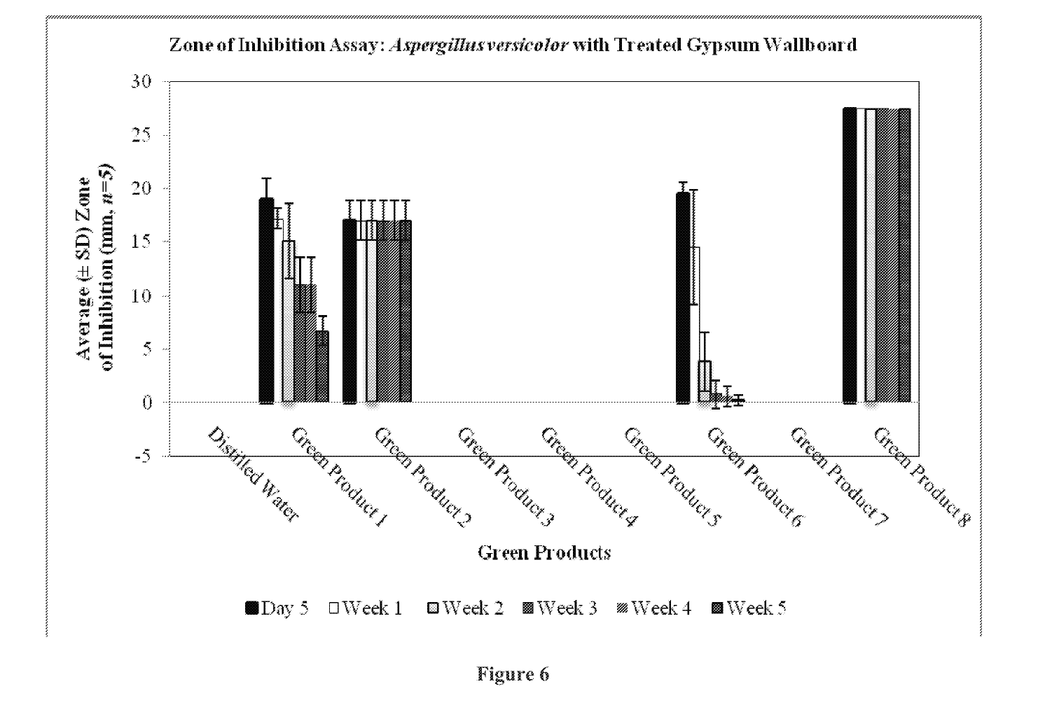

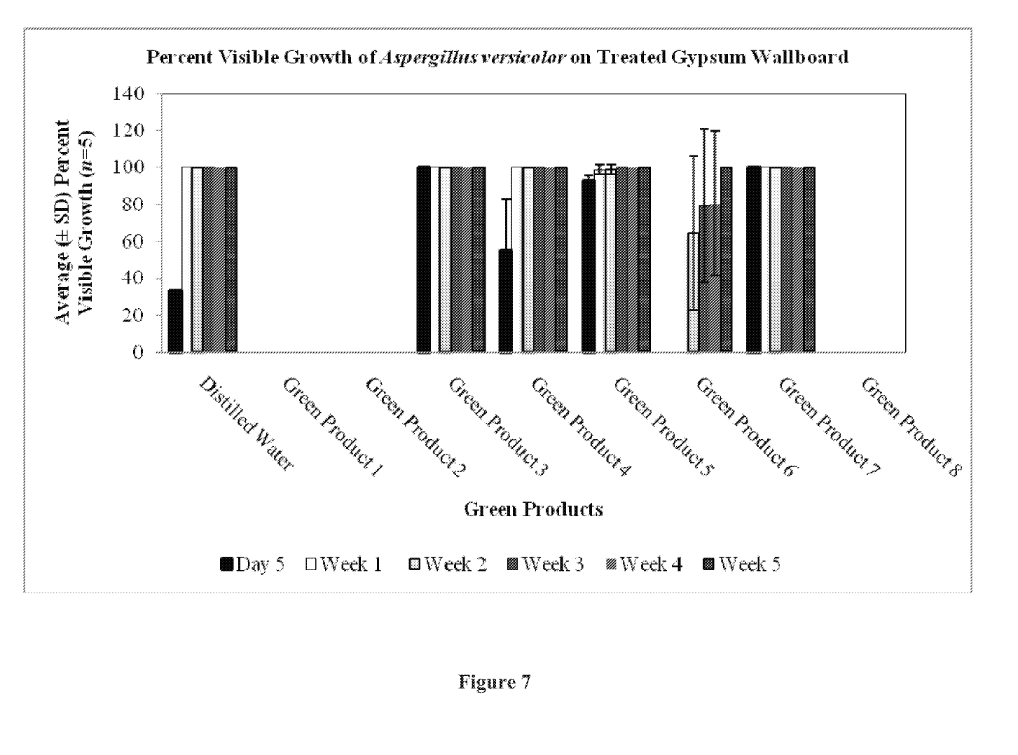

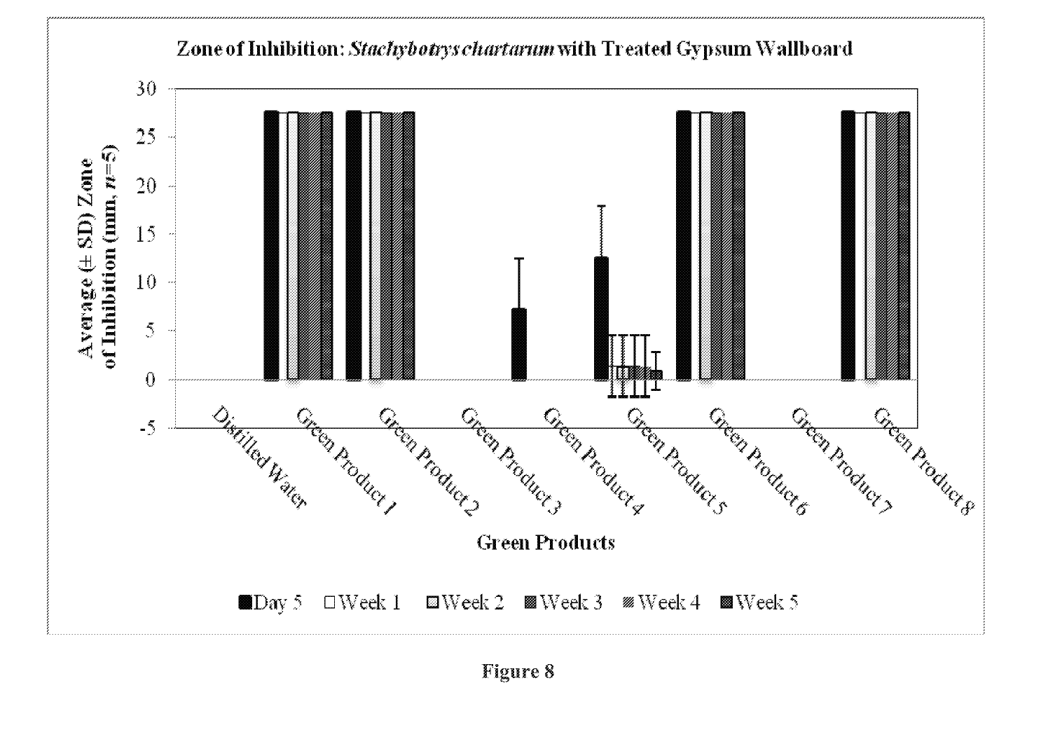

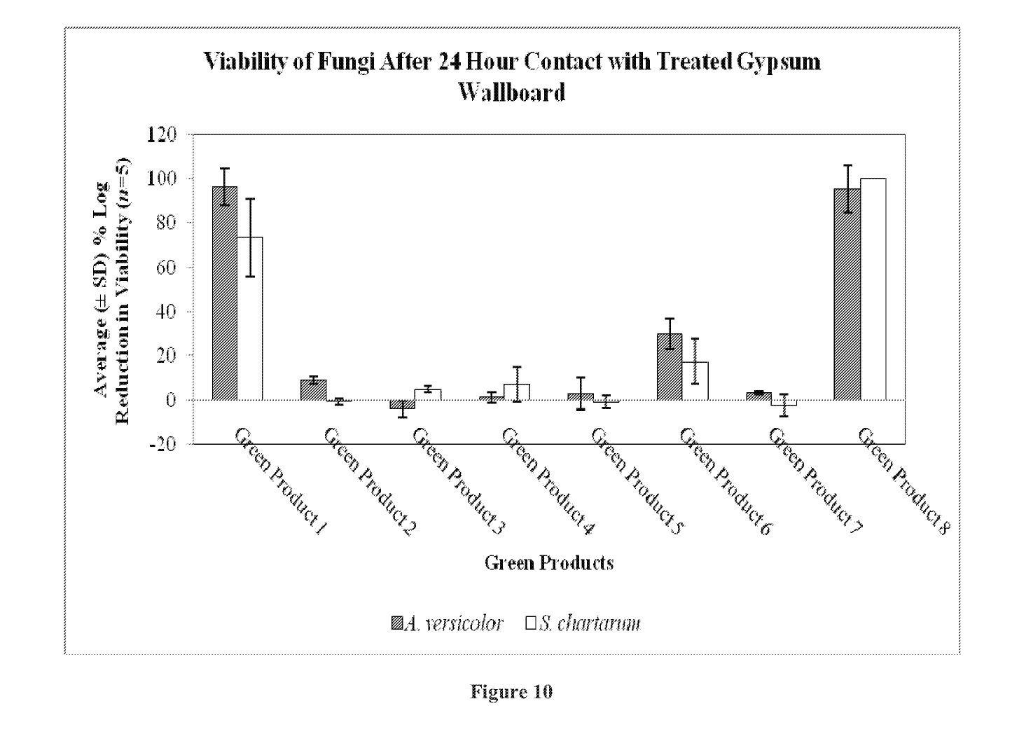

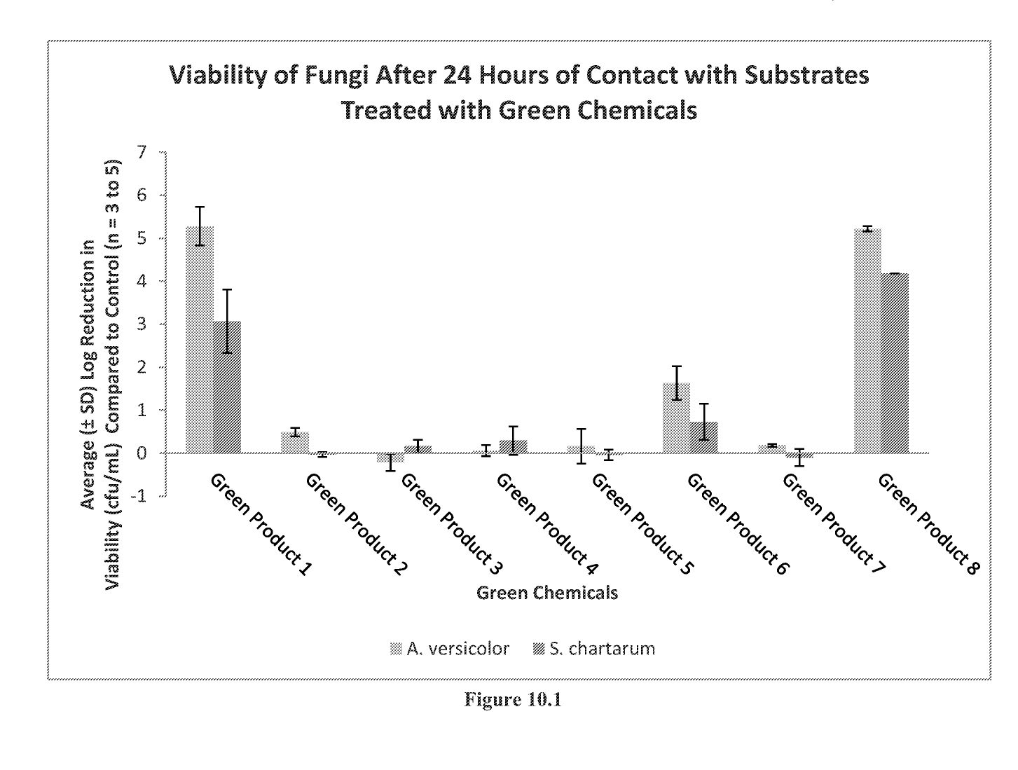

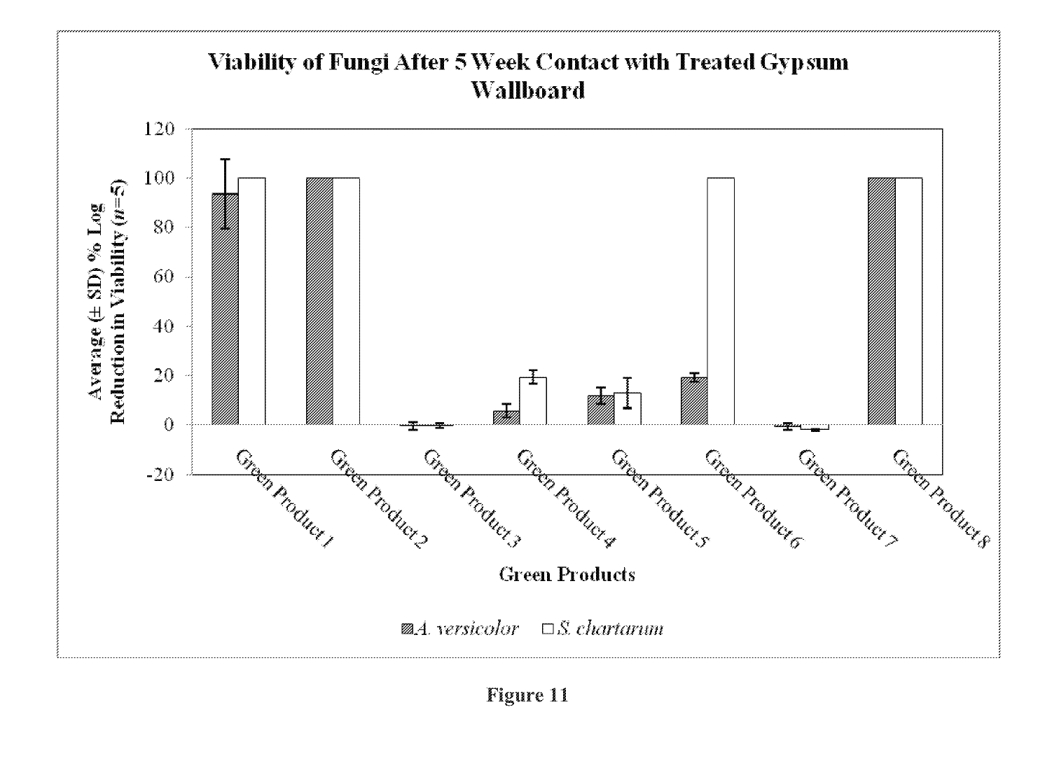

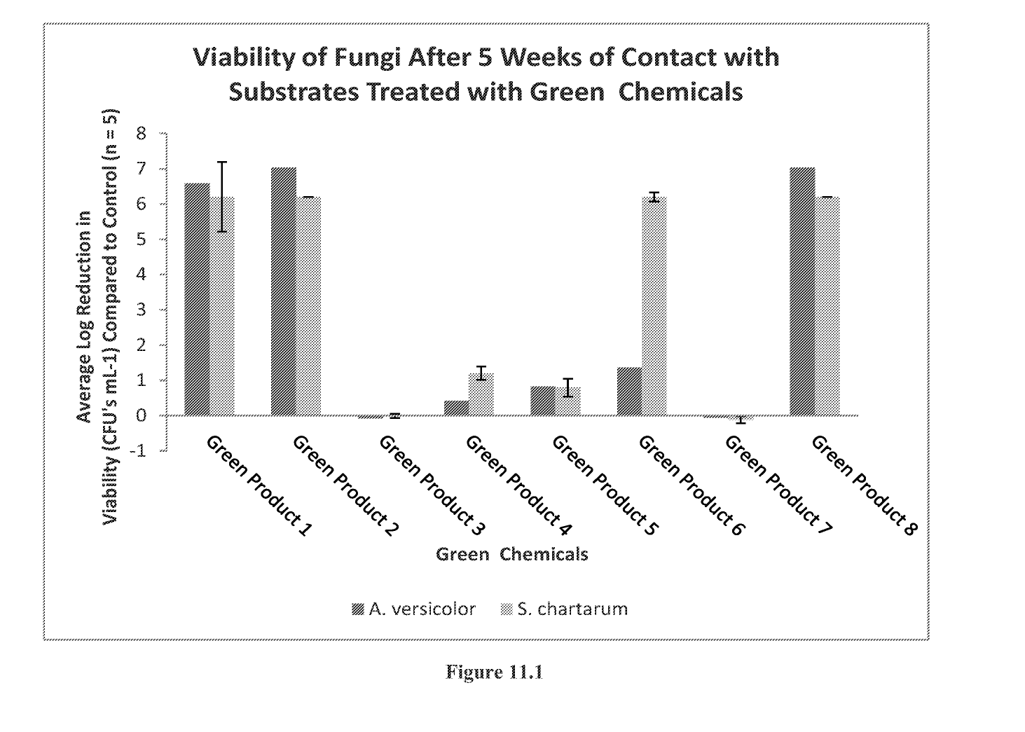

The fungicidal efficacies of the eight green test chemicals varied. Comparison of average percent log reductions in fungal viabilities after exposure of A. versicolor and S. chartarum fungi for 24 hours (FIG. 10) and 5 weeks (FIG. 11) to pretreated substrates showed that only green products 1 and 8 caused significant reduction in viability of the two fungi, following both short- and long-term incubation periods. Most importantly, the effect after a 5-week incubation period was 100% or nearly complete inhibition for the both fungi with no variation (p-value=>0.05). Stachybotrys chartarum was relatively more resistant compared to A. versicolor when the exposure was limited to 24 hours (p-value=<0.05). Further, products 1 and 8 showed no visible growth on the substrate surface for five weeks along with large zones of inhibition on nutritive agar indicating stability of active ingredients, with the exception of gradually decreasing ZOI of product 1 for A. versicolor (FIGS. 6-8). Green product 2, eliciting large stable zones of inhibition and complete inhibition of the visible growth on substrate surface, totally mitigated test fungi causing 100% reduction in the viability after 5 weeks of exposure. Nonetheless, this formulation was invariably ineffective after 24-hour of. Green product 6 was only marginally effective against one of the two test fungi in short term exposure. Further, a time-dependent decrease in ZOI concurrent with visible growth increasingly covering the entire substrate surface by week 5 was noted for A. versicolor. The decrease in viability for the two test fungi differed (p-value=<0.05) after 5 weeks of exposure to Green product 6. S. charatrum was more susceptible with 100% reduction in viability compared to marginal reduction for A. versicolor. Further, S. chartarum was completely inhibited with this formulation, consistent with stable zone of inhibition and no visible growth on substrate, unlike A. versicolor. Green products 3, 4, 5, and 7 showed unstable or no ZOI on agar and presence of visible growth on the substrate. These formulations were invariably ineffective in reducing the viabilities of the two test species, though decrease was slightly higher for products 4 and 5 after 5 weeks of exposure.

### Conclusions:

Not all green chemicals prevented colonization of Stachybotrys chartarum and Aspergillus versicolor on pretreated building substrates. Only two of eight tested formulations provided high or full (100%) short- and long-term protection against the test species. Fungistatic efficiencies were larger after 5 weeks of exposure for the other two formulations that were less effective in 24 hours of contact. Remaining products were variably ineffective. Differences were noted in susceptibility of the test genera to some of the test chemicals.

### Example 3

Methods applied are the same as those described for Examples 1 and 2 except where noted. Long term efficacy was determined after 8 weeks. Substrate Treatment: Six of the green antimicrobial products listed above (Table 6) were applied to substrates as follows. Gypsum wallboard (for A. alternata) or plywood (for P. brevicompactum), cut into 30 mm×30 mm sized substrates, were individually wrapped and autoclaved at 121° C. for 30 minutes. The sterile substrate substrates were then pretreated with antimicrobials by immersing the substrates in 20 milliliters of antimicrobial formulation in a Petri-dish for 10 minutes on each of the both sides. Treated substrates were used after draining the excess liquid. Substrate immersed in distilled water (control) served as the non-treated or negative control. V-8 juice agar was used to culture A. alternata and Potato Dextrose Agar as growth medium for P. brevicompactum.

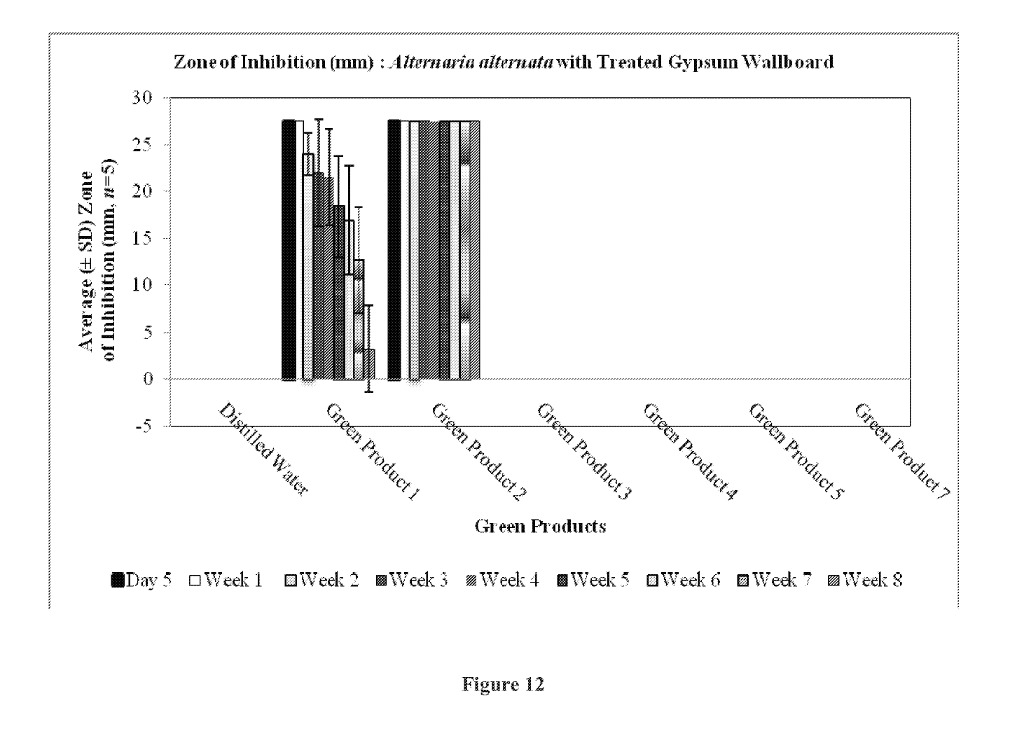

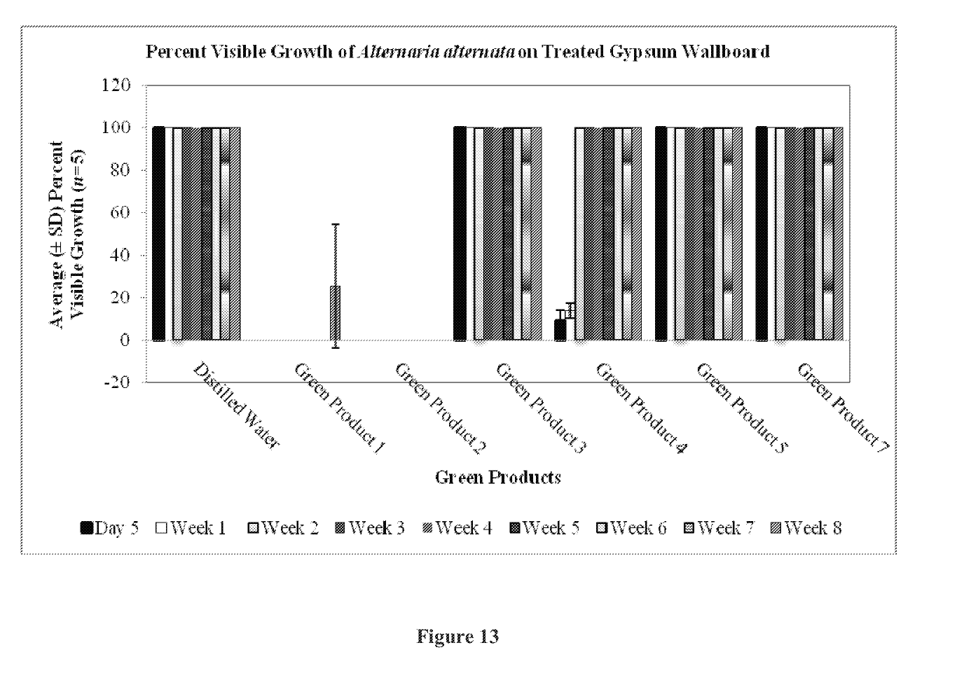

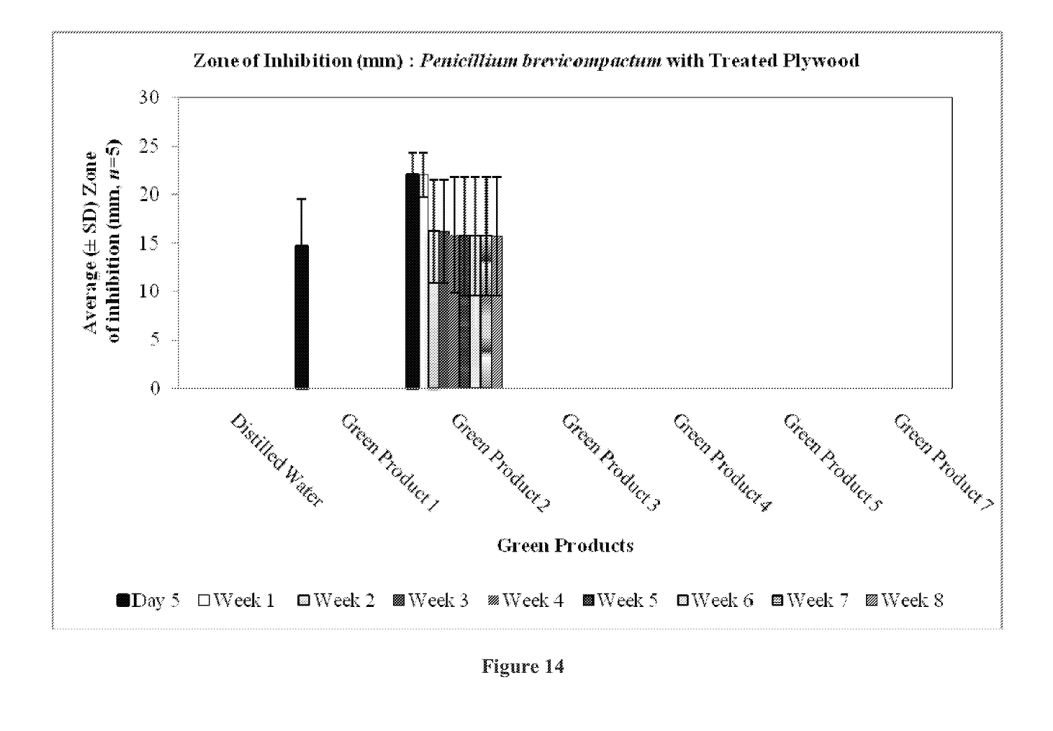

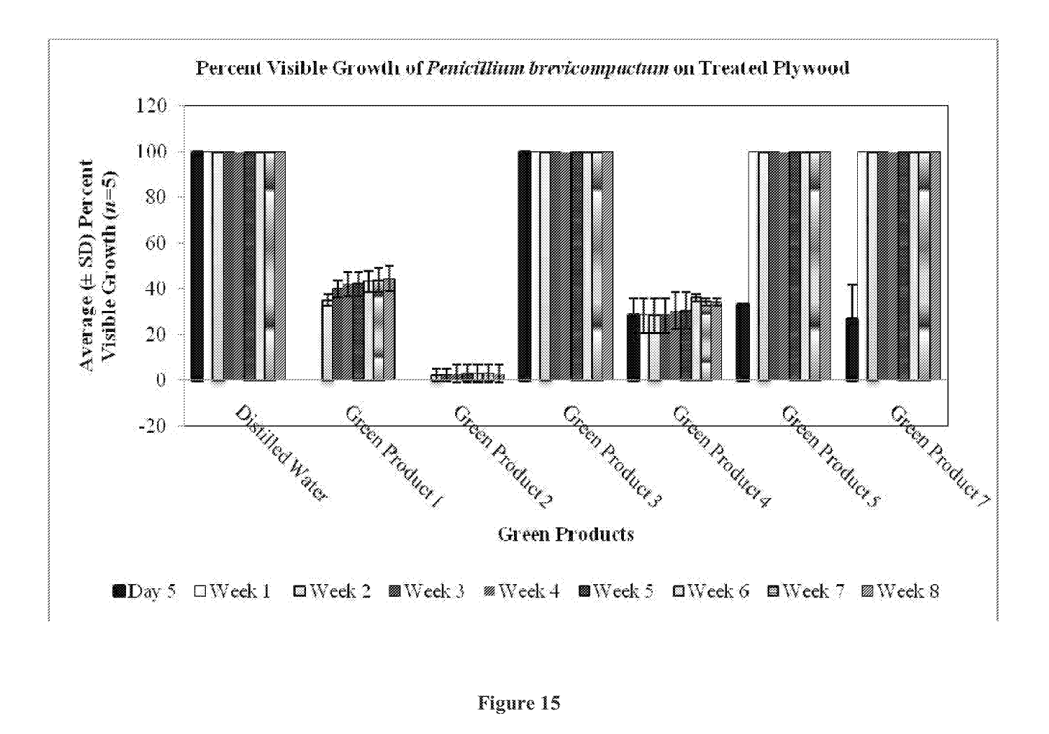

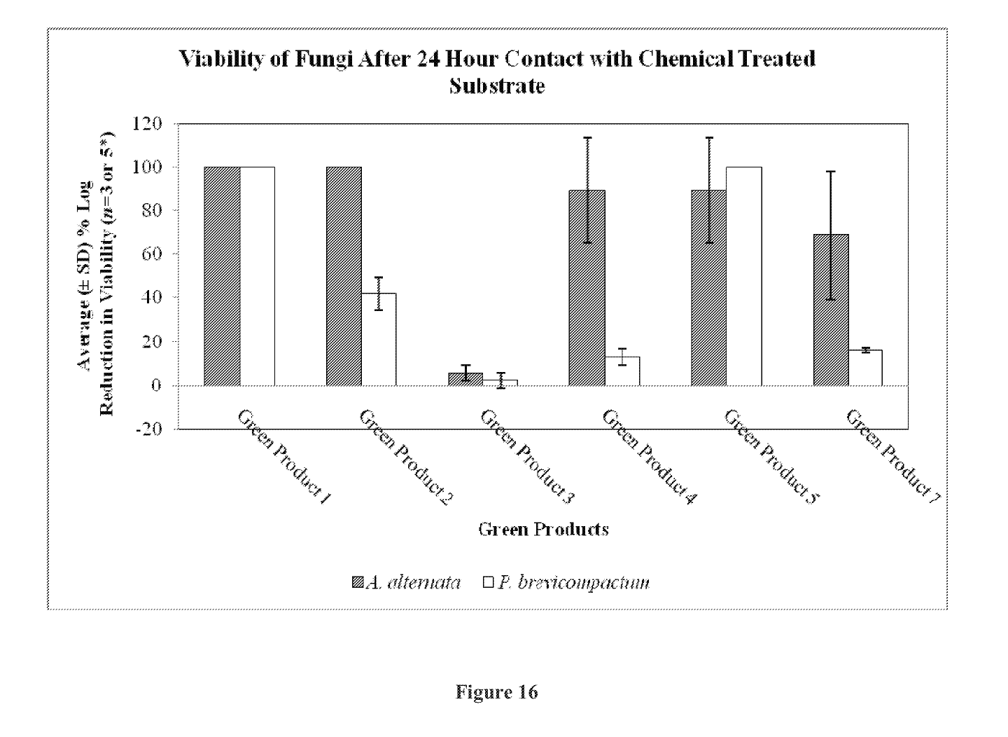

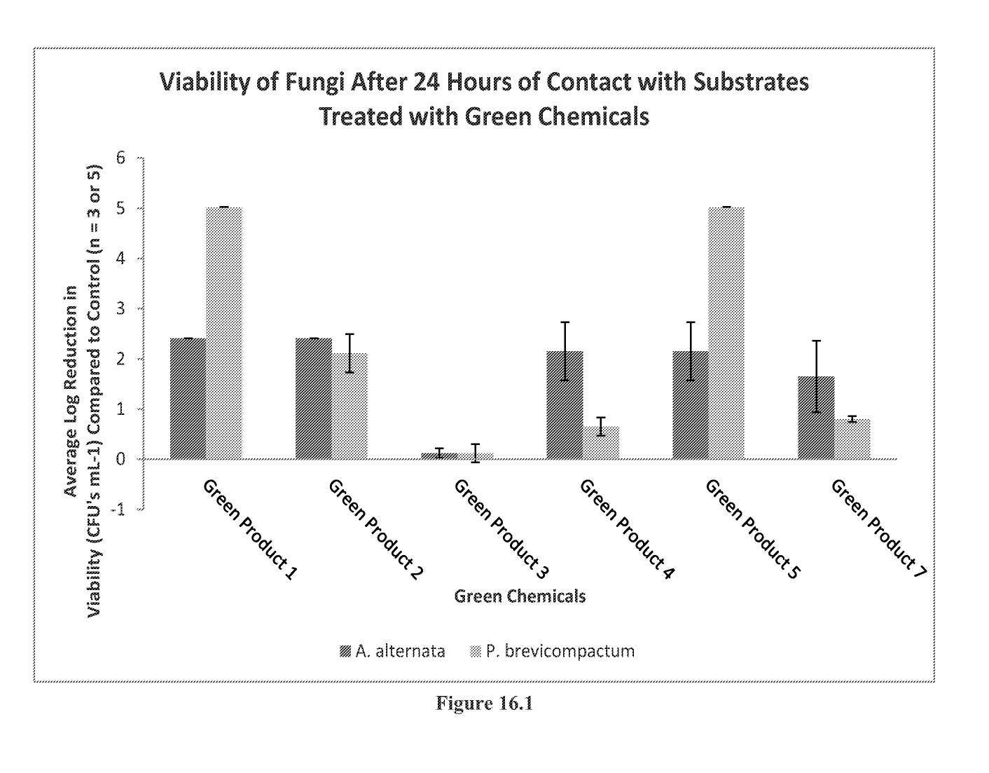

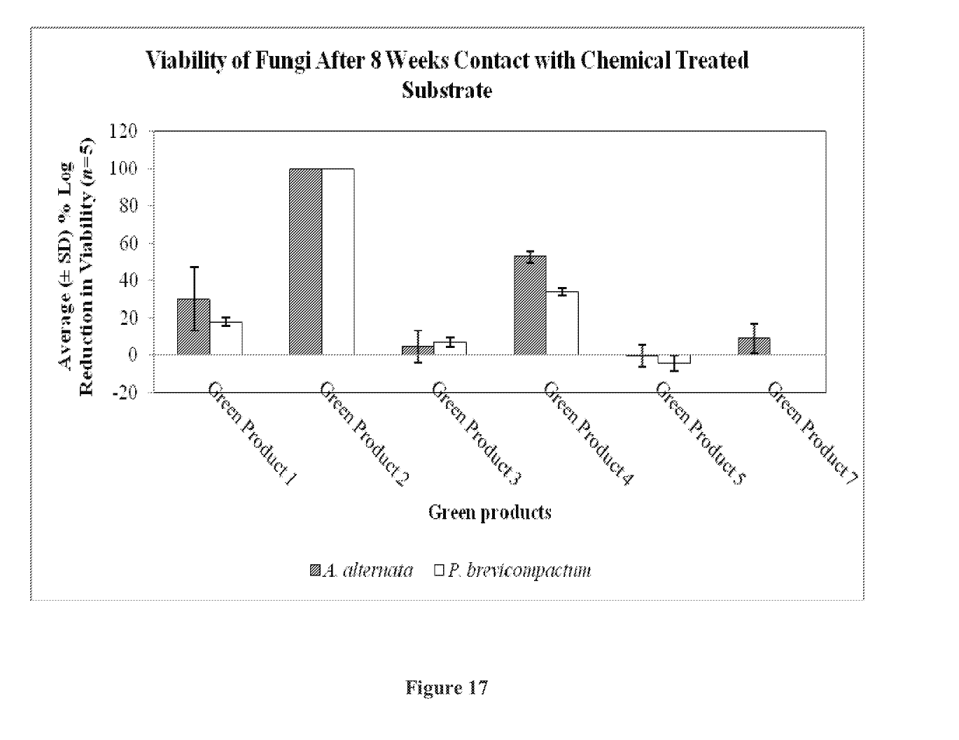

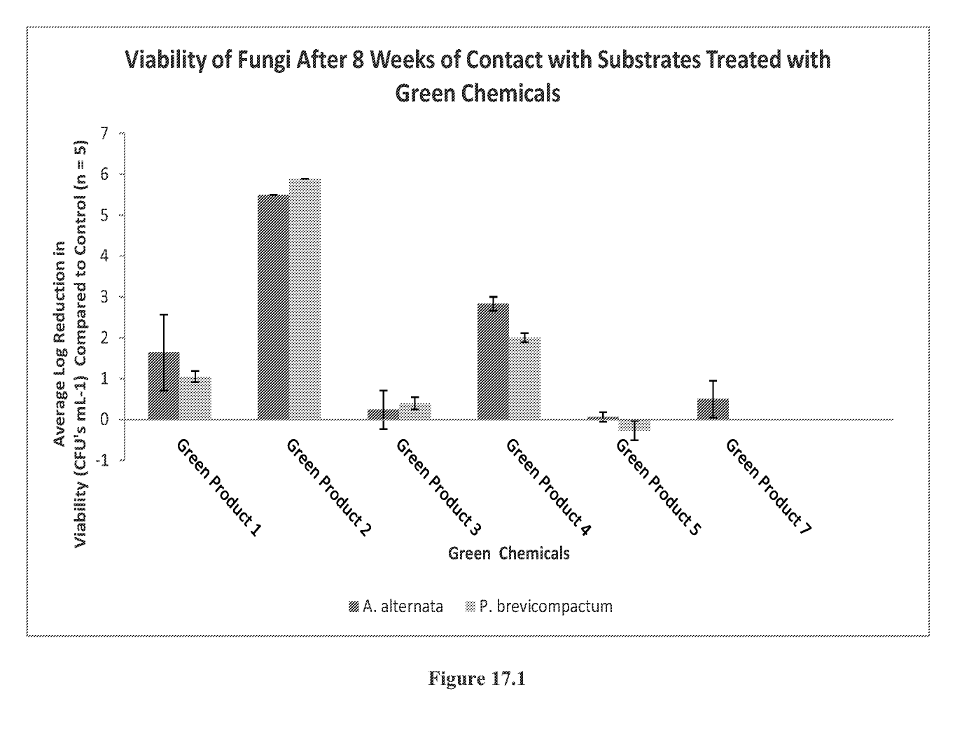

Green product 2 was the only green formulation of the six tested that showed large and relatively stable zone of inhibition on nutritive agar, in association with minimal to no visible growth of A. alternata and P. brevicompactum on the substrate surface post 8 weeks of exposure (FIGS. 12 to 15). Fungal viabilities were reduced by 100% with no difference in species response after incubation periods of this length (FIGS. 16 to 17), suggesting persistence of active ingredients. Nonetheless, this formulation caused variable decrease in the viabilities of A. alternata (100%) and P. brevicompactum (42%) in short-term exposure (p-value=<0.05), indicating that longer exposure period is necessary for the later fungus. Green product 1 treatment initially showed a ZOI coupled with reduced P. brevicompactum growth (<45%) on the substrate over an 8 week period (FIGS. 14 & 15). It also exhibited a steadily decreasing ZOI, allowing some growth of A. alternata on the substrate that was visible at week 8 (FIGS. 12 & 13). The full inhibitory effect (100% decreases in viability) was noted for both fungi subsequent to 24-hours of exposure to product 1. Nonetheless, as noted above, the reducing effect diminished over the 8-week period, indicative of the degradation of active ingredients, and fungus-specific variation in susceptibility (p-value=<0.05). Similarly, product 5 was only effective against test species when the exposure was short-term (average log reduction in 89%-100% range). Absence of ZOI, full coverage of substrate surface with visible growth by day 5, and no viability decrease post long-term exposure signaled rapid degradation of this formulation also. Green products 4 and 7 caused no ZOI and variable visible growth on the substrate surface and viability reductions differing in magnitude 24 hours post-exposure (p-value=<0.05). Alternaria alternata was less resistant (68%-89% range of average reduction in viability) compared to negligible mitigation of P. brevicompactum. The viability of both fungi was incompletely (<51%) decreased by the Green product 4 after 8 weeks of incubation, notwithstanding, significant difference in susceptibility among the two species (p-value=<0.05). Green product 7 was ineffective against A. alternata on long-term exposure, consistent with no ZOI and full 100% growth visible on the substrate as early as day 5. Data for P. brevicompactum is still awaited. Green product 3 was the only formulation with no inhibitory effect on any fungus either at short- or long-term periods of incubation.

### Conclusions:

Fungistatic action of the test chemicals varied. None of the formulations provided both short and long term protection against colonization of the test genera.

The discussion of the references herein is intended merely to summarize the assertions made by the authors and no admission is made that any reference constitutes prior art. Applicants reserve the right to challenge the accuracy and pertinence of the cited references.

1. A method of determining the effectiveness of an antimicrobial agent in reducing or inhibiting microbial growth on a substrate, the method comprising:
a) treating a substrate with the antimicrobial agent,
b) evenly dispersing a volume of a microbial suspension of known concentration on the top surface of the substrate,
c) placing the substrate in a sterile culture container,
d) incubating the substrate for one or more periods of time at a temperature and relative humidity that will allow growth of the microbe,
e) determining viable colony forming units by extracting viable microbes from the substrate and plating the extract on nutritive media and incubating the extract on nutrient media under conditions which allow growth of the microbe and,
f) comparing the viable colony forming units obtained from substrates treated with the antimicrobial agent to viable colony forming units obtained from substrates not treated with an antimicrobial agent or to colony forming units obtained from substrates treated with a different antimicrobial agent and subjected to steps a) through e).

2. The method of claim 1, wherein, incubating the substrate for one or more periods of time to determine efficacy of the antimicrobial, comprises incubating the substrate for a period of time determined to represent short term efficacy.

3. The method of claim 2, wherein the time determined to represent short term efficacy consists of about 24 hours.

4. The method of claim 1, wherein incubating the substrate for one or more periods of time to determine efficacy of the antimicrobial, comprises incubating the substrate for a period of time determined to represent long term efficacy.

5. The method of claim 4, wherein the time determined to represent long term efficacy is about 5 weeks.

6. A method of determining the effectiveness of an antimicrobial agent in reducing or inhibiting microbial growth on a substrate, the method comprising:
a) placing the substrate in the center of an agar plate, the agar plate comprising nutritive culture medium and inoculated with a microbial suspension of known concentration,
b) evenly dispersing a volume of the microbial suspension of known concentration on the top surface of the substrate,
c) incubating the substrate for period of time at a temperature and relative humidity that will allow growth of the microbe,
d) determining a zone of inhibition around the substrate by measuring the area of microbial growth inhibition around the substrate and,
e) comparing the zone of inhibition obtained from substrates treated with the antimicrobial agent to a zone of inhibition obtained from substrates not treated with a antimicrobial agent or to a zone of inhibition obtained from substrates treated with a different antimicrobial agent and subject to steps a) through d).

7. The method of claim 6, further comprising, determining microbial growth on the substrate surface, by
a) measuring the area of microbial growth on the substrate surface,
b) scoring the quality of the microbial growth using predetermined criteria and,
c) comparing the results to those of substrates not treated with the antimicrobial or substrates treated with a different antimicrobial agent and subject to the same conditions.

8. The method of claim 6, further comprising, determining viable colony forming units after incubation the substrate for one or more time periods, by extracting viable microbes from the substrate, plating the extract on appropriate nutritive media, incubating the extract under suitable growth conditions, and comparing the viable colony forming units to viable colony forming units of control samples maintained under similar conditions.

9. The method of claim 8, wherein incubating the substrate for one or more periods of time, comprises incubating the substrate for a period of time determined to represent short term efficacy.

10. The method of claim 9, wherein the time determined to represent short term efficacy consists of about 24 hours.

11. The method of claim 8, wherein, incubating the substrate for one or more periods of time to determine efficacy of the antimicrobial, comprises incubating the substrate for a period of time determined to represent long term efficacy.

12. The method of claim 11, wherein the time determined to represent long term efficacy is about 5 weeks.

13. A method of determining the effectiveness of an antimicrobial agent in reducing or inhibiting microbial growth on a substrate, the method comprising:
a) treating a substrate with the antimicrobial agent,
b) incubating the substrate for one or more periods of time, in the field, at a test site with an environment that will allow growth of one or more microbes,
c) determining viable colony forming units by extracting viable microbes from the substrate and plating the extract on nutritive media and under conditions which allow growth of the microbes and,
d) comparing the viable colony forming units obtained from the substrates treated with the antimicrobial agents to viable colony forming units obtained from substrates not treated with an antimicrobial agent or to viable colony forming units obtained from substrates treated with a different antimicrobial agent and subject to steps a) through c).

14. The method of claim 13, wherein incubating the substrate for one or more periods of time to determine efficacy of the antimicrobial, comprises incubating the substrate for a period of time determined to represent short term efficacy.

15. The method of claim 14, wherein the time determined to represent short term efficacy consists of about 24 hours.

16. The method of claim 13, wherein incubating the substrate for one or more periods of time to determine efficacy of the antimicrobial, comprises incubating the substrate for a period of time determined to represent long term efficacy.

17. The method of claim 16, wherein the time determined to represent long term efficacy is about 5 weeks.
